(19) United States
(12) Patent Application Publication
(10) Pub. No.: US 2002/0034785 A1
(43) Pub. Date: Mar. 21, 2002

(54) Calcitonin receptor

(76) Inventors: Daniel R. Soppet, Centreville, VA (US); Yi Li, Sunnyvale, CA (US); Steven M. Ruben, Olney, MD (US)

Correspondence Address: STERNE, KESSLER, GOLDSTEIN & FOX P.L.L.C., 1100 NEW YORK AVENUE, N.W., SUITE 600, WASHINGTON, DC, 20005-3934, US

(21) Appl. No.: 09/843,856

(22) Filed: Apr. 30, 2001

Related U.S. Application Data
(63) Continuation of application No. 08/970,758, filed on Nov. 14, 1997, now abandoned.
(60) Provisional application No. 60/030,934, filed on Nov. 15, 1996.

(51) Int. Cl. C07K 14/705; C07H 21/04; C12P 21/02; C12N 5/06

(52) U.S. Cl. 435/69.1; 435/325; 435/320.1; 530/350; 536/23.5

(57) ABSTRACT

The present invention relates to a novel calcitonin receptor protein which is a member of the G-protein couple receptor superfamily. In particular, isolated nucleic acid molecules are provided encoding the human calcitonin receptor protein. Calcitonin receptor polypeptides are also provided as are vectors, host cells and recombinant methods for producing the same. The invention further relates to screening methods for identifying agonists and antagonists of calcitonin receptor activity. Also disclosed are diagnostic assays for detecting diseases associated with altered calcitonin receptor expression.

This application claims the benefit of the filing date of provisional application No. 60/030,934 filed on Nov. 15, 1996, which is herein incorporated by reference.

### BACKGROUND OF THE INVENTION

1. Field of the Invention

The present invention relates to a novel human G-protein coupled receptor. More specifically, isolated nucleic acid molecules are provided encoding a human calcitonin receptor. Calcitonin receptor polypeptides are also provided, as are vectors, host cells and recombinant methods for producing the same. The invention further relates to screening methods for identifying agonists and antagonists of calcitonin receptor activity. Also disclosed are diagnostic assays for detecting diseases associated with altered calcitonin receptor expression.

2. Related Art

It is well established that many medically significant biological processes are mediated by proteins participating in signal transduction pathways that involve G-proteins and/or second messengers, e.g., cAMP (Lefkowitz, Nature, 351:353-354 (1991)). Herein these proteins are referred to as proteins participating in pathways with G-proteins or PPG proteins. Some examples of these proteins include the GPC receptors, such as those for adrenergic agents and dopamine (Kobilka, B. K., et al., PNAS, 84:46-50 (1987); Kobilka, B. K., et al., Science, 238:650-656 (1987); Bunzow, J. R., et al., Nature, 336:783-787 (1988)), G-proteins themselves, effector proteins, e.g., phospholipase C, adenyl cyclase, and phosphodiesterase, and actuator proteins, e.g., protein kinase A and protein kinase C (Simon, M. I., et al., Science, 252:802-808 (1991)).

For example, in one form of signal transduction, the effect of hormone binding is activation of an enzyme, adenylate cyclase, inside the cell. Enzyme activation by hormones is dependent on the presence of the nucleotide GTP, and GTP also influences hormone binding. A G-protein connects the hormone receptors to adenylate cyclase. G-protein was shown to exchange GTP for bound GDP when activated by hormone receptors. The GTP-carrying form then binds to an activated adenylate cyclase. Hydrolysis of GTP to GDP, catalyzed by the G-protein itself, returns the G-protein to its basal, inactive form, Thus, the G-protein serves a dual role, as an intermediate that relays the signal from receptor to effector, and as a clock that controls the duration of the signal.

The membrane protein gene superfamily of G-protein coupled receptors has been characterized as having seven putative transmembrane domains. The domains are believed to represent transmembrane α-helices connected by extracellular or cytoplasmic loops. G-protein coupled receptors include a wide range of biologically active receptors, such as hormone, viral, growth factor and neuroreceptors.

G-protein coupled receptors have been characterized as including these seven conserved hydrophobic stretches of about 20 to 30 amino acids, connecting at least eight divergent hydrophilic loops. The G-protein family of coupled receptors includes dopamine receptors which bind neuroleptic drugs used for treating psychotic and neurological disorders. Other examples of members of this family include calcitonin, adrenergic, endothelin, cAMP, adenosine, muscarinic, acetylcholine, serotonin, histamine, thrombin, kinin, follicle stimulating hormone, opsins, endothelial differentiation gene-1 receptor, rhodopsins, odorant, cytomegalovirus receptors, etc.

Most G-protein coupled receptors have single conserved cysteine residues in each of the first two extracellular loops which form disulfide bonds that are believed to stabilize functional protein structure. The 7 transmembrane regions are designated as TM1, TM2, TM3, TM4, TM5, TM6, and TM7. TM3 has been implicated in signal transduction.

Phosphorylation and lipidation (palmitylation or farnesylation) of cysteine residues can influence signal transduction of some G-protein coupled receptors. Most G-protein coupled receptors contain potential phosphorylation sites within the third cytoplasmic loop and/or the carboxy terminus. For several G-protein coupled receptors, such as the β-adrenoreceptor, phosphorylation by protein kinase A and/or specific receptor kinases mediates receptor desensitization.

For some receptors, the ligand binding sites of G-protein coupled receptors are believed to comprise a hydrophilic socket, formed by several G-protein coupled receptors transmembrane domains, that is surrounded by hydrophobic residues of the G-protein coupled receptors. The hydrophilic side of each G-protein coupled receptor transmembrane helix is postulated to face inward and form the polar ligand binding site. TM3 has been implicated in several G-protein coupled receptors as having a ligand binding site, including the TM3 aspartate residue. Additionally, TM5 serines, a TM6 asparagine and TM6 or TM7 phenylalanines or tyrosines are also implicated in ligand binding.

G-protein coupled receptors can be intracellularly coupled by heterotrimeric G-proteins to various intracellular enzymes, ion channels and transporters (see, Johnson et al., Endoc., Rev., 10:317-331 (1989)). Different G-protein α-subunits preferentially stimulate particular effectors to modulate various biological functions in a cell. Phosphorylation of cytoplasmic residues of G-protein coupled receptors have been identified as an important mechanism for the regulation of G-protein coupling of some G-protein coupled receptors. G-protein coupled receptors are found in numerous sites within a mammalian host.

Over the past 15 years, nearly 150 therapeutic agents targeting 7 transmembrane (7 TM) receptors have been successfully introduced onto the market. This indicates that these receptors have an established, proven history as therapeutic targets. Clearly there is a need for identification and characterization of further receptors which can play a role in preventing, ameliorating or correcting dysfunctions or diseases, including, but not limited to, pain, cancers, anorexia, bulimia, asthma, Parkinson's disease, acute heart failure, hypotension, hypertension, urinary retention, osteoporosis, angina pectoris, myocardial infarction, ulcers, asthma, allergies, benign prostatic hypertrophy and psychotic and neurological disorders, including anxiety, schizophrenia, manic depression, delirium, dementia or severe mental retardation, dyskinesias, such as Huntington's disease or Gilles dela Tourett's syndrome, among others.

### SUMMARY OF THE INVENTION

The present invention provides isolated nucleic acid molecules comprising a polynucleotide encoding the calcitonin receptor having the amino acid sequence shown in FIGS. 1A and B (SEQ ID NO:2) or the amino acid sequence encoded by the cDNA clone deposited in a bacterial host as ATCC Deposit Number 97730 on Sep. 23, 1996.

The present invention also relates to recombinant vectors, which include the isolated nucleic acid molecules of the present invention, and to host cells containing the recombinant vectors, as well as to methods of making such vectors and host cells and for using them for production of calcitonin receptor polypeptides or peptides by recombinant techniques.

The invention further provides an isolated calcitonin polypeptide having an amino acid sequence encoded by a polynucleotide described herein. The present invention also provides a screening method for identifying compounds capable of enhancing or inhibiting (i.e., agonists or antagonists) a cellular response induced by the calcitonin receptor.,

The invention further provides methods for isolating antibodies that bind specifically to a calcitonin receptor polypeptide having an amino acid sequence as described herein. Such antibodies are useful diagnostically or therapeutically as describe below.

G-protein coupled receptors, such as the calcitonin receptor of the present invention, play a role a number of dysfunctions or diseases. Thus, agonists or antagonists of the calcitonin receptor of the present invention could be used therapeutically to prevent, ameliorate or correct dysfunctions or diseases associated with the calcitonin receptor of the present invention. Another aspect of the invention is related to methods for treating an individual in need of an increased or decreased level of calcitonin receptor activity in the body.

### BRIEF DESCRIPTION OF THE FIGURES

FIGS. 1A and B show the nucleotide (SEQ ID NO:1) and deduced amino acid (SEQ ID NO:2) sequences of human calcitonin receptor. The protein has a deduced molecular weight of about 61287 Da.

FIG. 2 shows the predicted location of each of the seven transmembrane domains of the calcitonin receptor.

### DETAILED DESCRIPTION

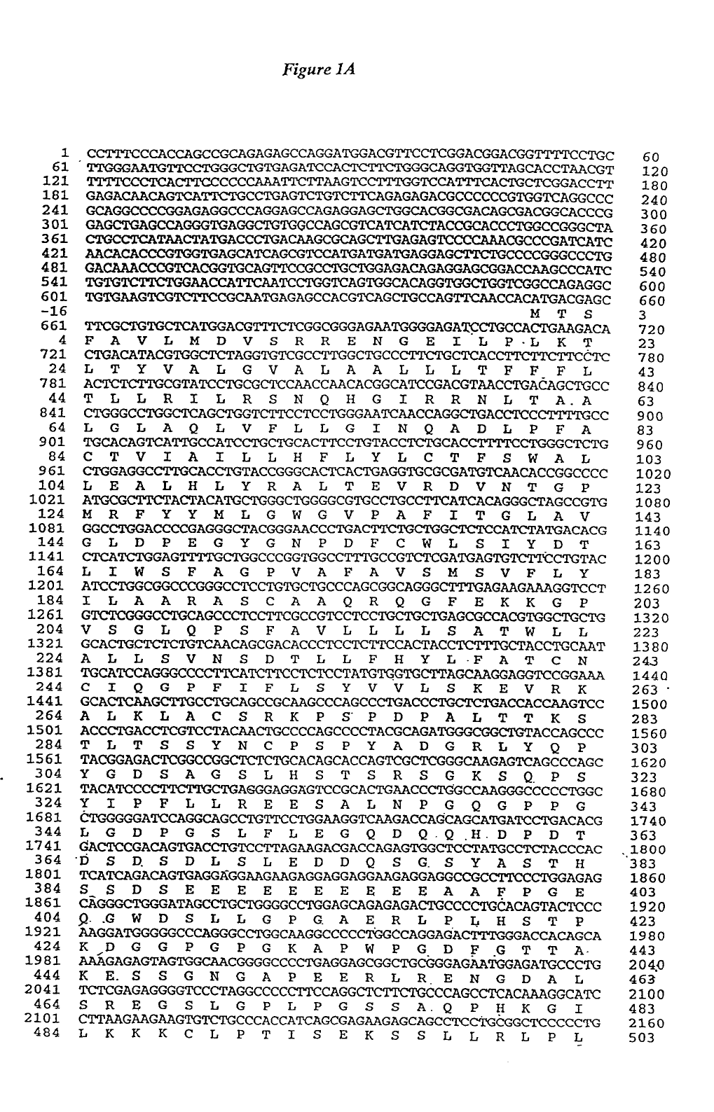

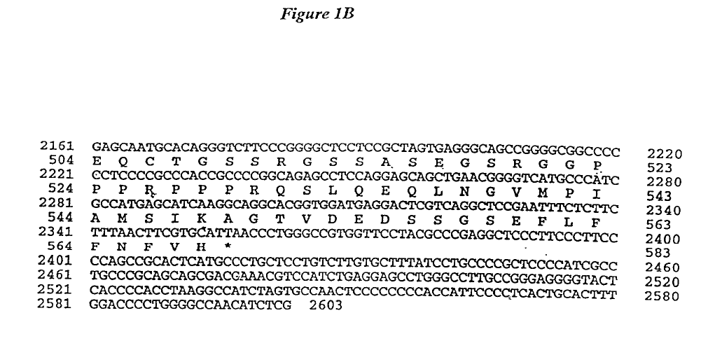

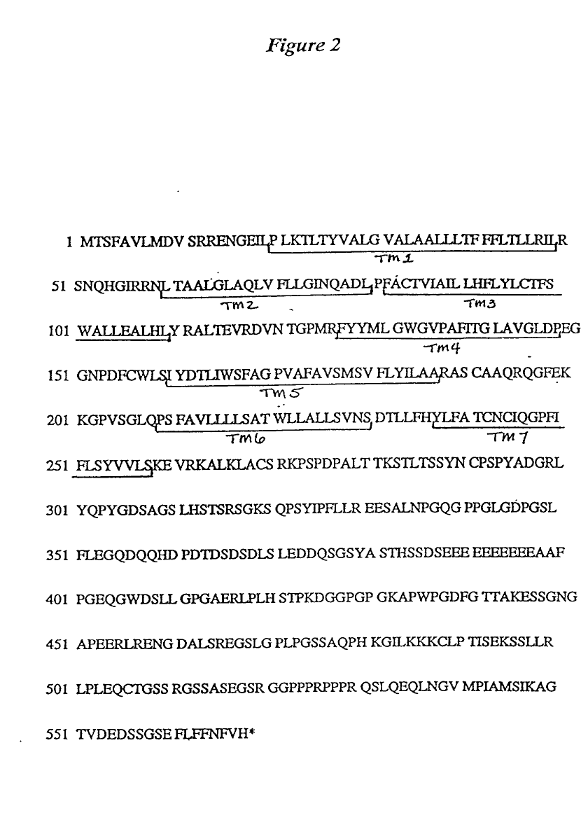

The present invention provides isolated nucleic acid molecules comprising a polynucleotide encoding a calcitonin receptor polypeptide having the amino acid sequence shown in FIGS. 1A and B (SEQ ID NO:2), which was determined by sequencing a cloned cDNA. The calcitonin receptor protein of the present invention shares sequence homology with rat calcitonin receptor A, rat calcitonin receptor B, rat growth hormone releasing hormone receptor GHRH, and hormone receptor EMR1 (SEQ ID NOs:3-6). The nucleotide sequence shown in FIGS. 1A and B (SEQ ID NO: 1) was obtained by sequencing the HCEPR64 clone, which was deposited on Sep. 23, 1996 at the American Type Culture Collection, 12301 Park Lawn Drive, Rockville, Md. 20852, and given accession number 97730. The deposited clone is contained in the pBluescript SK(-) plasmid (Stratagene, La Jolla, Calif.).

Nucleic Acid Molecules

Unless otherwise indicated, all nucleotide sequences determined by sequencing a DNA molecule herein were determined using an automated DNA sequencer (such as the Model 373 from Applied Biosystems, Inc.), and all amino acid sequences of polypeptides encoded by DNA molecules determined herein were predicted by translation of a DNA sequence determined as above. Therefore, as is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion.

Using the information provided herein, such as the nucleotide sequence in FIGS. 1A and B, a nucleic acid molecule of the present invention encoding a calcitonin receptor polypeptide may be obtained using standard cloning and screening procedures, such as those for cloning cDNAs using mRNA as starting material. Illustrative of the invention, the nucleic acid molecule described in FIGS. 1A and B (SEQ ID NO:1) was discovered in a eDNA library derived from cerebellum using the expressed sequence tag (EST) analysis (Adams, et al. Science 252:1651-1656(1991); Adams et al. Nature 355:632-634(1992); Adams et al. Nature 377 Supp, 3-174 (1995)). The gene was also identified in cDNA libraries from the following tissues cerebellum, early brain, fetal brain and uterine cancer. The determined nucleotide sequence of the calcitonin receptor cDNA of FIGS. 1A and B (SEQ ID NO:1) contains an open reading frame encoding a protein of 568 amino acid residues, with an initiation codon at positions 652-654 of the nucleotide sequence in FIGS. 1A and B (SEQ ID NO:1), and a deduced molecular weight of about 61287 Da. The calcitonin receptor protein shown in FIGS. 1A and B (SEQ ID NO:2) is about 21% identical to rat calcitonin receptor A, is about 21% identical to rat calcitonin receptor B, is about 25% identical to rat growth hormone releasing hormone receptor GHRH, and is about 31% identical to hormone receptor EMR1.

As one of ordinary skill would appreciate, due to the possibilities of sequencing errors discussed above, the actual calcitonin receptor polypeptide encoded by the deposited cDNA comprises about 568 amino acids, but may be anywhere in the range of 555-575 amino acids.

As indicated, nucleic acid molecules of the present invention may be in the form of RNA, such as mRNA, or in the form of DNA, including, for instance, cDNA and genomic DNA obtained by cloning or produced synthetically. The DNA may be double-stranded or single-stranded. Single-stranded DNA or RNA may be the coding strand, also known as the sense strand, or it may be the non-coding strand, also referred to as the anti-sense strand.

By “isolated” nucleic acid molecule(s) is intended a nucleic acid molecule, DNA or RNA, which has been removed from its native environment For example, recombinant DNA molecules contained in a vector are considered isolated for the purposes of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. Isolated nucleic acid molecules according to the present invention further include such molecules produced synthetically.

Isolated nucleic acid molecules of the present invention include DNA molecules comprising an open reading frame (ORF) shown in FIGS. 1A and B (SEQ ID NO: 1); DNA molecules comprising the coding sequence for the calcitonin receptor protein shown in FIGS. 1A and B (568 amino acids) (SEQ ID NO:2); and DNA molecules which comprise a sequence substantially different from those described above but which, due to the degeneracy of the genetic code, still encode the calcitonin receptor protein. Of course, the genetic code is well known in the art. Thus, it would be routine for one skilled in the art to generate the degenerate variants described above.

In addition, the invention provides nucleic acid molecules having nucleotide sequences related to extensive portions of SEQ ID NO: 1 which have been determined from the following related cDNA clones: HCEPR64R (SEQ ID NO:13), and HCETE57R (SEQ ID NO:14).

The sequence of a public ESTs also having nucleotide sequences related to extensive portions of SEQ ID NO:1, are the following GenBank Accession Nos: R60111 (SEQ ID NO:15), R60049 (SEQ ID NO: 16), AA326354 (SEQ ID NO:17), W52864 (SEQ ID NO:18), AA037392 (SEQ ID NO:19), T06233 (SEQ ID NO:20), AA477598 (SEQ ID NO:21), F13341 (SEQ ID NO:22), AA477470 (SEQ ID NO:23), T77092 (SEQ ID NO:24), H55526 (SEQ ID NO:25), and AA349458 (SEQ ID NO:26).

In another aspect, the invention provides isolated nucleic acid molecules encoding the calcitonin receptor polypeptide having an amino acid sequence encoded by the cDNA clone contained in the plasmid deposited as ATCC Deposit No. 97730 on Sep. 23, 1996. The invention further provides an isolated nucleic acid molecule having the nucleotide sequence shown in FIGS. 1A and B (SEQ ID NO: 1) or the nucleotide sequence of the calcitonin receptor cDNA contained in the above-described deposited clone, or a nucleic acid molecule having a sequence complementary to one of the above sequences. Such isolated molecules, particularly DNA molecules, are useful as probes for gene mapping, by in situ hybridization with chromosomes, and for detecting expression of the calcitonin receptor gene in human tissue, for instance, by Northern blot analysis.

The present invention is further directed to fragments of the isolated nucleic acid molecules described herein. By a fragment of an isolated nucleic acid molecule having the nucleotide sequence of the deposited cDNA or the nucleotide sequence shown in FIGS. 1A and B (SEQ ID NO: 1) is intended fragments at least about 15 nt, and more preferably at least about 20 nt, still more preferably at least about 30 nt, and even more preferably, at least about 40 nt in length which are useful as diagnostic probes and primers as discussed herein. Of course, larger fragments 50, 75, 100, 125, 150, 175,200, 225, 250, 275, 300, 325, 350, 375, 400, 425, 450, 475, 500, 525, 550, 575, 600, 625, 650, 675, 700, 725, 750,775,800, 825,850,875,900,925, 950,975,1000, 1025, 1050, 1075, 1100, 1125, 1150,1175, 1200, 1225, 1250, 1275, 1300, 1325, 1350, 1375, 1400, 1425, 1450, 1475 or 1500 nt in length are also useful according to the present invention as are fragments corresponding to most, if not all, of the nucleotide sequence of the deposited cDNA or as shown in FIGS. 1A and B (SEQ ID NO:1). By a fragment at least 20 nt in length, for example, is intended fragments which include 20 or more contiguous bases from the nucleotide sequence of the deposited cDNA or the nucleotide sequence as shown in FIGS. 1A and B (SEQ ID NO:1).

Preferred nucleic acid fragments of the present invention include nucleic acid molecules encoding epitope-bearing portions of the calcitonin receptor protein that were predicted from the results of an “Antigenic Index-Jameson-Wolf” graph. In particular, such nucleic acid fragments of the present invention include nucleic acid molecules encoding: a polypeptide comprising amino acid residues from about 10 to about 20 in SEQ ID NO:2; a polypeptide comprising amino acid residues from about 49 to about 60 in SEQ ID NO:2; a polypeptide comprising amino acid residues from about 113 to about 123 in SEQ ID NO:2; a polypeptide comprising amino acid residues from about 145 to about 154 in SEQ ID NO:2; a polypeptide comprising amino acid residues from about 189 to about 209 in SEQ ID NO:2; and a polypeptide comprising amino acid residues from about 259 to about 560 in SEQ ID NO:2. The inventors have determined that the above polypeptide fragments are antigenic regions of the calcitonin receptor protein. Methods for determining other such epitope-bearing portions of the calcitonin receptor protein are described in detail below.

In another aspect, the invention provides an isolated nucleic acid molecule comprising a polynucleotide which hybridizes under stringent hybridization conditions to a portion of the polynucleotide in a nucleic acid molecule of the invention described above, for instance, the cDNA clone contained in ATCC Deposit No. 97730. By “stringent hybridization conditions” is intended overnight incubation at 42° C. in a solution comprising: 50% formamide, 5× SSC (150 mM NaCl, 15 mM trisodium citrate), 50 mM sodium phosphate (pH 7.6), 5× Denhardt's solution, 10% dextran sulfate, and 20 g/ml denatured, sheared salmon sperm DNA, followed by washing the filters in 0.1× SSC at about 65° C.

By a polynucleotide which hybridizes to a “portion” of a polynucleotide is intended a polynucleotide (either DNA or RNA) hybridizing to at least about 15 nucleotides (nt), and more preferably at least about 20 nt, still more preferably at least about 30 nt, and even more preferably about 30-70 nt of the reference polynucleotide. These are useful as diagnostic probes and primers as discussed above and in more detail below.

By a portion of a polynucleotide of “at least 20 nt in length,” for example, is intended 20 or more contiguous nucleotides from the nucleotide sequence of the reference polynucleotide (e.g., the deposited cDNA or the nucleotide sequence as shown in FIGS. 1A and B (SEQ ID NO: 1)). Of course, a polynucleotide which hybridizes only to a poly A sequence (such as the 3′ terminal poly(A) tract of the calcitonin receptor cDNA shown in FIGS. 1A and B (SEQ ID NO: 1)), or to a complementary stretch of T (or U) resides, would not be included in a polynucleotide of the invention used to hybridize to a portion of a nucleic acid of the invention, since such a polynucleotide would hybridize to any nucleic acid molecule containing a poly (A) stretch or the complement thereof (e.g., practically any double-stranded eDNA clone).

As indicated, nucleic acid molecules of the present invention which encode a calcitonin receptor polypeptide may include, but are not limited to those encoding the coding sequence for the polypeptide and additional sequences, such as a pre-, or pro- or prepro- protein sequence; the coding sequence of the polypeptide, with or without the aforementioned additional coding sequences, together with additional, non-coding sequences, including for example, but not limited to introns and non-coding 5′ and 3′ sequences, such as the transcribed, non-translated sequences that play a role in transcription, mRNA processing, including splicing and polyadenylation signals, for example—ribosome binding and stability of mRNA; an additional coding sequence which codes for additional amino acids, such as those which provide additional functionalities. Thus, the sequence encoding the polypeptide may be fused to a marker sequence, such as a sequence encoding a peptide which facilitates purification of the fused polypeptide. In certain preferred embodiments of this aspect of the invention, the marker amino acid sequence is a hexa-histidine peptide, such as the tag provided in a pQE vector (Qiagen, Inc.), among others, many of which are commercially available. As described in Gentz et al., Proc. Natl. Acad. Sci. USA 86:821-824 (1989), for instance, hexa-histidine provides for convenient purification of the fusion protein. The “HA” tag is another peptide useful for purification which corresponds to an epitope derived from the influenza hemagglutinin protein, which has been described by Wilson et al., Cell 37: 767 (1984). As discussed below, other such fusion proteins include the calcitonin receptorfused to Fc at the N- or C-terminus.

The present invention further relates to variants of the nucleic acid molecules of the present invention, which encode portions, analogs or derivatives of the calcitonin receptor protein. Variants may occur naturally, such as a natural allelic variant. By an “allelic variant” is intended one of several alternate forms of a gene occupying a given locus on a chromosome of an organism. Genes II, Lewin, B., ed., John Wiley & Sons, New York (1985). Non-naturally occurring variants may be produced using art-known mutagenesis techniques.

Such variants include those produced by nucleotide substitutions, deletions or additions, which may involve one or more nucleotides. The variants may be altered in coding regoins, non-coding regions, or both. Alterations in the coding regions may produce conservative or non-conservative amino acid substitutions, deletions or additions. Especially preferred among these are silent substitutions, additions and deletions, which do not alter the properties and activities of the calcitonin receptor protein or portions thereof. Also especially preferred in this regard are conservative substitutions.

Further embodiments of the invention include isolated nucleic acid molecules comprising a polynucleotide having a nucleotide sequence at least 90% identical, and more preferably at least 95%, 96%, 97%, 98% or 99% identical to (a) a nucleotide sequence encoding the calcitonin receptor polypeptide having the amino acid sequence in SEQ ID NO:2; (b) a nucleotide sequence encoding the calcitonin receptor polypeptide having the amino acid sequence in SEQ ID NO:2, but lacking the N-terminal methionine; (c) a nucleotide sequence encoding the calcitonin receptor polypeptide having the amino acid sequence encoded by the cDNA clone contained in ATCC Deposit No. 97730; (d) a nucleotide sequence encoding a polypeptide comprising the calcitonin receptor extracellular regions selected from the group consisting of: amino acid residues from about 1 to about 18, amino acid residues from about 148 to about 159, amino acid residues from about 188 to about 209, and amino acid residues from about 231 to about 236 in SEQ ID NO:2; (e) a nucleotide sequence encoding a polypeptide comprising the calcitonin receptor intracellular regions selected from the group consisting of:amino acid residues from about 50 to about 59, amino acid residues from about 110 to about 125, amino acid residues from about 188 to about 209, and amino acid residues from about 259 to about 560 in SEQ ID NO:2; (f) a nucleotide sequence encoding a polypeptide comprising the calcitonin receptor transmembrane domain 1 (amino acid residues from about 20 to about 49 in SEQ ID NO:2); (g) a nucleotide sequence encoding a polypeptide comprising the calcitonin receptor transmembrane domain 2 (amino acid residues from about 60 to about 80 in SEQ ID NO:2); (h) a nucleotide sequence encoding a polypeptide comprising the calcitonin receptor transmembrane domain 3 (amino acid residues from about 82 to about 109 in SEQ ID NO:2); (i) a nucleotide sequence encoding a polypeptide comprising the calcitonin receptor transmembrane domain 4 (amino acid residues from about 126 to about 147 in SEQ ID NO:2); (j) a nucleotide sequence encoding the calcitonin receptor transmembrane domain 5 (amino acid residues from about 160 to about 187 in SEQ ID NO:2); (k) a nucleotide sequence encoding a polypeptide comprising the calcitonin receptor transmembrane domain 6 (amino acid residues from about 209 to about 230 in SEQ ID NO:2); (1) a nucleotide sequence encoding a polypeptide comprising the calcitonin receptor transmembrane domain 7 (amino acid residues from about 237 to about 258 in SEQ ID NO:2); and (m) a nucleotide sequence complementary to any of the nucleotide sequences in (a), (b), (c), (d), (e), (f), (g), (h), (i), (j), (k) or (l), above.

By a polynucleotide having a nucleotide sequence at least, for example, 95% “identical” to a reference nucleotide sequence encoding a calcitonin receptorpolypeptide is intended that the nucleotide sequence of the polynucleotide is identical to the reference sequence except that the polynucleotide sequence may include up to five point mutations per each 100 nucleotides of the reference nucleotide sequence encoding the calcitonin receptorpolypeptide. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 90%, 95%, 96%, 97%, 98% or 99% identical to, for instance, the nucleotide sequence shown in FIGS. 1A and B or to the nucleotides sequence of the deposited cDNA clone can be determined conventionally using known computer programs such as the Bestfit program (Wisconsin Sequence Analysis Package, Version 8 for Unix, Genetics Computer Group, University Research Park, 575 Science Drive, Madison, Wis. 53711. Bestfit uses the local homology algorithm of Smith and Waterman, Advances in Applied Mathematics 2: 482-489 (1981), to find the best segment of homology between two sequences. When using Bestfit or any other sequence alignment program to determine whether a particular sequence is, for instance, 95% identical to a reference sequence according to the present invention, the parameters are set, of course, such that the percentage of identity is calculated over the full length of the reference nucleotide sequence and that gaps in homology of up to 5% of the total number of nucleotides in the reference sequence are allowed.

The present application is directed to nucleic acid molecules at least 90%, 95%, 96%, 97%, 98% or 99% identical to the nucleic acid sequence shown in FIGS. 1A and B (SEQ ID NO:1) or to the nucleic acid sequence of the deposited cDNA, irrespective of whether they encode a polypeptide having calcitonin receptor activity. This is because even where a particular nucleic acid molecule does not encode a polypeptide having calcitonin receptor activity, one of skill in the art would still know how to use the nucleic acid molecule, for instance, as a hybridization probe or a polymerase chain reaction (PCR) primer. Uses of the nucleic acid molecules of the present invention that do not encode a polypeptide having calcitonin receptor activity include, inter alia, (1) isolating the calcitonin receptor gene or allelic variants thereof in a cDNA library; (2) in situ hybridization (e.g., “FISH”) to metaphase chromosomal spreads to provide precise chromosomal location of the calcitonin receptor gene, as described in Verma et al., Human Chromosomes. A Manual of Basic Techniques, Pergamon Press, New York (1988); and Northern Blot analysis for detecting calcitonin receptor mRNA expression in specific tissues.

Preferred, however, are nucleic acid molecules having sequences at least 90%, 95%, 96%, 97%, 98% or 99% identical to the nucleic acid sequence shown in FIGS. 1A and B (SEQ ID NO: 1) or to the nucleic acid sequence of the deposited cDNA which do, in fact, encode a polypeptide having calcitonin receptor protein activity. By “a polypeptide having calcitonin receptor activity” is intended polypeptides exhibiting activity similar, but not necessarily identical, to an activity of the calcitonin receptor protein of the invention, as measured in a particular biological assay.

One can evaluate whether a candidate polypeptide has calcitonin receptor activity by performing simple binding kinetics to measure receptor affinity for a particular ligand (i.e., calcitonin). Binding kinetic analysis experiments are well known to those skilled in the art (Chizzonite et al., Proc Natl. Acad. Sci. 86:8029-8033 (1989); Mizel, et al., J. Immunol. 138:2906-2912 (1987)). One can monitor the presence of calcitonin receptor activity by testing whether the putative calcitonin receptor polypeptide has a similar binding affinity for a particular ligand as the receptor of the present invention. The receptor of the present invention can serve as a reference for the assay for receptor activity.

Of course, due to the degeneracy of the genetic code, one of ordinary skill in the art will immediately recognize that a large number of the nucleic acid molecules having a sequence at least 90%, 95%, 96%, 97%, 98%, or 99% identical to the nucleic acid sequence of the deposited cDNA or the nucleic acid sequence shown in FIGS. 1A and B (SEQ ID NO: 1) will encode a polypeptide “having calcitonin receptor protein activity.” In fact, since degenerate variants of these nucleotide sequences all encode the same polypeptide, this will be clear to the skilled artisan even without performing the above described comparison assay. It will be further recognized in the art that, for such nucleic acid molecules that are not degenerate variants, a reasonable number will also encode a polypeptide having calcitonin receptor protein activity. This is because the skilled artisan is fully aware of amino acid substitutions that are either less likely or not likely to significantly effect protein function (e.g., replacing one aliphatic amino acid with a second aliphatic amino acid).

For example, guidance concerning how to make phenotypically silent amino acid substitutions is provided in Bowie, J. U. et al., “Deciphering the Message in Protein Sequences: Tolerance to Amino Acid Substitutions,” Science 247:1306-1310 (1990), wherein the authors indicate that proteins are surprisingly tolerant of amino acid substitutions.

Vectors and Host Cells

The present invention also relates to vectors which include the isolated DNA molecules of the present invention, host cells which are genetically engineered with the recombinant vectors, and the production of calcitonin receptor polypeptides or fragments thereof by recombinant techniques.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The DNA insert should be operatively linked to an appropriate promoter, such as the phage lambda PL promoter, the E. coli lac, trp and tac promoters, the SV40 early and late promoters and promoters of retroviral LTRs, to name a few. Other suitable promoters will be known to the skilled artisan. The expression constructs will further contain sites for transcription initiation, termination and, in the transcribed region, a ribosome binding site for translation. The coding portion of the transcripts expressed by the constructs will preferably include a translation initiating at the beginning and a termination codon (UAA, UGA or UAG) appropriately positioned at the end of the polypeptide to be translated.

As indicated, the expression vectors will preferably include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS and Bowes melanoma cells; and plant cells. Appropriate culture mediums and conditions for the above-described host cells are known in the art.

Among vectors preferred for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Among preferred eukaryotic vectors are pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986).

The polypeptide may be expressed in a modified form, such as a fusion protein, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art. A preferred fusion protein comprises a heterologous region from immunoglobulin that is useful to solubilize proteins. For example, EP-A-O 464 533 (Canadian counterpart 2045869) discloses fusion proteins comprising various portions of constant region of immunoglobin molecules together with another human protein or part thereof. In many cases, the Fc part in a fusion protein is thoroughly advantageous for use in therapy and diagnosis and thus results, for example, in improved pharmacokinetic properties (EP-A 0232 262). On the other hand, for some uses it would be desirable to be able to delete the Fc part after the fusion protein has been expressed, detected and purified in the advantageous manner described. This is the case when Fc portion proves to be a hindrance to use in therapy and diagnosis, for example when the fusion protein is to be used as antigen for immunizations. In drug discovery, for example, human proteins, such as, hIL5-receptor has been fused with Fc portions for the purpose of high-throughput screening assays to identify antagonists of hIL-5. See, D. Bennett et al., Journal of Molecular Recognition, Vol. 8:52-58 (1995) and K. Johanson et al., The Journal of Biological Chemistry, Vol. 270, No. 16:9459-9471 (1995).

The calcitonin receptor protein can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification. Polypeptides of the present invention include naturally purified products, products of chemical synthetic procedures, and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes.

Calcitonin Receptor Polypeptides and Fragments

The invention further provides an isolated calcitonin receptor having the amino acid sequence encoded by the deposited cDNA, or the amino acid sequence in FIGS. 1A and B (SEQ ID NO:2), or a peptide or polypeptide comprising a portion of the above polypeptides.

It will be recognized in the art that some amino acid sequences of the calcitonin receptor polypeptide can be varied without significant effect of the structure or function of the protein. If such differences in sequence are contemplated, it should be remembered that there will be critical areas on the protein which determine activity.

Thus, the invention further includes variations of the calcitonin receptor polypeptide which show substantial calcitonin receptor activity or which include regions of calcitonin receptor protein such as the protein portions discussed below. Such mutants include deletions, insertions, inversions, repeats, and type substitutions. As indicated above, guidance concerning which amino acid changes are likely to be phenotypically silent can be found in Bowie, J. U., et al., “Deciphering the Message in Protein Sequences: Tolerance to Amino Acid Substitutions,” Science 247:1306-1310 (1990).

Thus, the fragment, derivative or analog of the polypeptide of FIGS. 1A and B (SEQ ID NO:2), or that encoded by the deposited eDNA, may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the polypeptide is fused with another compound, such as a compound to increase the half-life of the polypeptide (for example, polyethylene glycol), or (iv) one in which the additional amino acids are fused to the polypeptide, such as an IgG Fc fusion region peptide or leader or secretory sequence or a sequence which is employed for purification of the mature polypeptide or a proprotein sequence. Such fragments, derivatives and analogs are deemed to be within the scope of those skilled in the art from the teachings herein.

Of particular interest are substitutions of charged amino acids with another charged amino acid and with neutral or negatively charged amino acids. The latter results in proteins with reduced positive charge to improve the characteristics of the calcitonin protein. The prevention of aggregation is highly desirable. Aggregation of proteins not only results in a loss of activity but can also be problematic when preparing pharmaceutical formulations, because they can be immunogenic. (Pinckard et al., Clin Exp. Immunol. 2:331-340 (1967); Robbins et al., Diabetes 36:838-845 (1987); Cleland et al. Crit. Rev. Therapeutic Drug Carrier Systems 10:307-377 (1993)).

As indicated, changes are preferably of a minor nature, such as conservative amino acid substitutions that do not significantly affect the folding or activity of the protein (see Table 1).

TABLE 1
Conservative Amino Acid Substitutions.

| | |
|---|---|
| Aromatic | Phenylalanine |
| | Tryptophan |
| | Tyrosine |
| Hydrophobic | Leucine |
| | Isoleucine |
| | Valine |
| Polar | Glutamine |
| | Asparagine |
| Basic | Arginine |
| | Lysine |
| | Histidine |
| Acidic | Aspartic Acid |
| | Glutamic Acid |
| Small | Alanine |
| | Serine |
| | Threonine |
| | Methionine |
| | Glycine |

Of course, the number of amino acid substitutions a skilled artisan would make depends on many factors, including those described above. Generally speaking, the number of substitutions for any given calcitonin receptor polypeptide will not be more than 50, 40, 30, 25, 20, 15, 10, 5 or 3.

Amino acids in the calcitonin receptor protein of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity such as receptor binding or in vitro, or in vitro proliferative activity. Sites that are critical for ligand-receptor binding can also be determined by structural analysis such as crystallization, nuclear magnetic resonance or photoaffinity labeling (Smith et al., J. Mol. Biol. 224:899-904 (1992) and de Vos et al. Science 255:306-312 (1992)).

The polypeptides of the present invention are preferably provided in an isolated form, and preferably are substantially purified. By “isolated polypeptide” is intended a polypeptide removed from its native environment. Thus, a polypeptide produced and/or contained within a recombinant host cell is considered isolated for purposes of the present invention. Also intended as an “isolated polypeptide” are polypeptides that have been purified, partially or substantially, from a recombinant host cell. For example, a recombinantly produced version of the calcitonin receptor polypeptide can be substantially purified by the one-step method described in Smith and Johnson, Gene 67:31-40 (1988).

The polypeptides of the present invention include the polypeptide encoded by the deposited cDNA, the polypeptide encoded by the deposited cDNA minus the N-terminal methionine, a polypeptide comprising amino acids about 1 to about 568 in SEQ ID NO:2, a polypeptide comprising amino acids about 2 to about 568 in SEQ ID NO:2, the extracellular regions, the intracellular regions, transmembrane domain 1, transmembrane domain 2, transmembrane domain 3, transmembrane domain 4, transmembrane domain 5, transmembrane domain 6, transmembrane domain 7, as well as polypeptides which are at least 80% identical, more preferably at least 90% or 95% identical, still more preferably at least 96%, 97%, 98% or 99% identical to the polypeptide encoded by the deposited cDNA, to the polypeptide of FIGS. 1A and B (SEQ ID NO:2), and also include portions of such polypeptides with at least 30 amino acids and more preferably at least 50 amino acids. The present invention also includes deletion of the transmembrane region only and retention of at least part of the intracellular domain itself or fusion with at least part of an alternate intracellular domain as described in WO96/04382.

By a polypeptide having an amino acid sequence at least, for example, 95% “identical” to a reference amino acid sequence of a calcitonin receptor polypeptide is intended that the amino acid sequence of the polypeptide is identical to the reference sequence except that the polypeptide sequence may include up to five amino acid alterations per each 100 amino acids of the reference amino acid of the calcitonin receptor polypeptide. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 90%, 95%, 96%, 97%, 98% or 99% identical to, for instance, the amino acid sequence shown in FIGS. 1A and B (SEQ ID NO:2) or to the amino acid sequence encoded by deposited cDNA clone can be determined conventionally using known computer programs such the Bestfit program (Wisconsin Sequence Analysis Package, Version 8 for Unix, Genetics Computer Group, University Research Park, 575 Science Drive, Madison, Wis. 53711. When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

The polypeptide of the present invention could be used as a molecular weight marker on SDS-PAGE gels or on molecular sieve gel filtration columns using methods well known to those of skill in the art.

In another aspect, the invention provides a peptide or polypeptide comprising an epitope-bearing portion of a polypeptide of the invention. The epitope of this polypeptide portion is an immunogenic or antigenic epitope of a polypeptide described herein. An “immunogenic epitope” is defined as a part of a protein that elicits an antibody response when the whole protein is the immunogen. On the other hand, a region of a protein molecule to which an antibody can bind is defined as an “antigenic epitope.” The number of immunogenic epitopes of a protein generally is less than the number of antigenic epitopes. See, for instance, Geysen et al., Proc. Natl. Acad. Sci. USA 81:3998-4002 (1983).

As to the selection of peptides or polypeptides bearing an antigenic epitope (i.e., that contain a region of a protein molecule to which an antibody can bind), it is well known in that art that relatively short synthetic peptides that mimic part of a protein sequence are routinely capable of eliciting an antiserum that reacts with the partially mimicked protein. See, for instance, Sutcliffe, J. G., Shinnick, T. M., Green, N. and Learner, R. A. (1983). Antibodies that react with predetermined sites on proteins. Science 219:660-666. Peptides capable of eliciting protein-reactive sera are frequently represented in the primary sequence of a protein, can be characterized by a set of simple chemical rules, and are confined neither to immunodominant regions of intact proteins (i.e., immunogenic epitopes) nor to the amino or carboxyl terminals.

Antigenic epitope-bearing peptides and polypeptides of the invention are therefore useful to raise antibodies, including monoclonal antibodies, that bind specifically to a polypeptide of the invention.

Antigenic epitope-bearing peptides and polypeptides of the invention preferably contain a sequence of at least seven, more preferably at least nine and most preferably at least between about 15 to about 30 amino acids contained within the amino acid sequence of a polypeptide of the invention.

Non-limiting examples of antigenic polypeptides or peptides that can be used to generate calcitonin receptor-specific antibodies include:a polypeptide comprising amino acid residues from about 10 to about 20 in SEQ ID NO:2; a polypeptide comprising amino acid residues from about 49 to about 60 in SEQ ID NO:2; a polypeptide comprising amino acid residues from about 113 to about 123 in SEQ ID NO:2; a polypeptide comprising amino acid residues from about 145 to about 154 in SEQ ID NO:2; a polypeptide comprising amino acid residues from about 189 to about 209 in SEQ ID NO:2; and a polypeptide comprising amino acid residues from about 259 to about 560 in SEQ ID NO:2.

The epitope-bearing peptides and polypeptides of the invention may be produced by any conventional means. Houghten, R. A. (1985) General method for the rapid solid-phase synthesis of large numbers of peptides: specificity of antigen-antibody interaction at the level of individual amino acids. Proc. Natl. Acad. Sci. USA 82:5131-5135. This “Simultaneous Multiple Peptide Synthesis (SMPS)” process is further described in U.S. Pat. No. 4 631,211 to Houghten et al. (1986).

As one of skill in the art will appreciate, calcitonin receptor polypeptides of the present invention and the epitope-bearing fragments thereof described above can be combined with parts of the constant domain of immunoglobulins (IgG), resulting in chimeric polypeptides. These fusion proteins facilitate purification and show an increased half-life in vivo. This has been shown, e.g., for chimeric proteins consisting of the first two domains of the human CD4-polypeptide and various domains of the constant regions of the heavy or light chains of mammalian immunoglobulins (EPA 394,827; Traunecker et al., Nature 331:84-86 (1988)). Fusion proteins that have a disulfide-linked dimeric structure due to the IgG part can also be more efficient in binding and neutralizing other molecules than the monomeric calcitonin receptor protein or protein fragment alone (Fountoulakis et al., J Biochem 270:3958-3964 (1995)).

Polynucleotide Assays

This invention is also related to the use of the calcitonin receptor polynucleotides to detect complementary polynucleotides such as, for example, as a diagnostic reagent. Detection of a mutated form of calcitonin receptor associated with a dysfunction will provide a diagnostic tool that can add or define a diagnosis of a disease or susceptibility to a disease which results from under-expression or over-expression or altered expression of calcitonin receptor by assaying the expression level of the gene encoding the calcitonin receptor protein. In addition, individuals carrying mutations in the human calcitonin receptor gene may be detected at the DNA level by a variety of techniques. Nucleic acids for diagnosis may be obtained from a biological sample. The genomic DNA may be used directly for detection or may be amplified enzymatically by using PCR prior to analysis. PCR (Saiki et al., Nature, 324: 163-166 (1986)). RNA or cDNA may also be used in the same ways. As an example, PCR primers complementary to the nucleic acid encoding calcitonin receptor can be used to identify and analyze calcitonin receptor expression and mutations. For example, deletions and insertions can be detected by a change in size of the amplified product in comparison to the normal genotype. Point mutations can be identified by hybridizing amplified DNA to radiolabeled calcitonin receptor RNA or alternatively, radiolabeled calcitonin receptor antisense DNA sequences. Perfectly matched sequences can be distinguished from mismatched duplexes by RNase A digestion or by differences in melting temperatures.

By “assaying the expression level of the gene encoding the calcitonin receptor protein” is intended qualitatively or quantitatively measuring or estimating the level of the calcitonin receptor protein or the level of the mRNA encoding the calcitonin receptor protein in a first biological sample either directly (e.g., by determining or estimating absolute protein level or mRNA level) or relatively (e.g., by comparing to the calcitonin receptor protein level or mRNA level in a second biological sample).

Thus, the invention provides a diagnostic method useful during diagnosis of such disorders, which involves: (a) assaying calcitonin receptor gene expression level in cells or body fluid of an individual; (b) comparing the calcitonin receptor gene expression level with a standard calcitonin receptor gene expression level, whereby an increase or decrease in the assayed calcitonin receptor gene expression level compared to the standard expression level is indicative of such disorders. As will be appreciated in the art, once a standard calcitonin receptor protein level or mRNA level is known, it can be used repeatedly as a standard for comparison.

By “biological sample” is intended any biological sample obtained from an individual, cell line, tissue culture, or other source which contains calcitonin receptor protein or mRNA. Biological samples include mammalian body fluids (such as sera, plasma, urine, synovial fluid and spinal fluid) which contain calcitonin receptor protein, and ovarian, prostate, heart, placenta, pancreas liver, spleen, lung, breast and umbilical tissue.

Sequence differences between a reference gene and genes having mutations also may be revealed by direct DNA sequencing. In addition, cloned DNA segments may be employed as probes to detect specific DNA segments. The sensitivity of such methods can be greatly enhanced by appropriate use of PCR or another amplification method. For example, a sequencing primer is used with double-stranded PCR product or a single-stranded template molecule generated by a modified PCR. The sequence determination is performed by conventional procedures with radiolabeled nucleotide or by automatic sequencing procedures with fluorescent-tags.

Genetic testing based on DNA sequence differences may be achieved by detection of alteration in electrophoretic mobility of DNA fragments in gels, with or without denaturing agents. Small sequence deletions and insertions can be visualized by high resolution gel electrophoresis. DNA fragments of different sequences may be distinguished on denaturing formamide gradient gels in which the mobilities of different DNA fragments are retarded in the gel at different positions according to their specific melting or partial melting temperatures (see, e.g., Myers et al., Science, 230: 1242 (1985)).

Sequence changes at specific locations also may be revealed by nuclease protection assays, such as RNase and S1 protection or the chemical cleavage method (e.g., Cotton et al., Proc. Natl. Acad. Sci., USA, 85: 4397-4401 (1985)).

Thus, the detection of a specific DNA sequence may be achieved by methods such as hybridization, RNase protection, chemical cleavage, direct DNA sequencing or the use of restriction enzymes, (e.g., restriction fragment length polymorphisms (“RFLP”) and Southern blotting of genomic DNA. Total cellular RNA can be isolated from a biological sample using any suitable technique such as the single-step guanidinium-thiocyanate-phenol-chloroform method described in Chomczynski and Sacchi, Anal. Biochem. 162:156-159 (1987). Levels of mRNA encoding the calcitonin receptor protein are then assayed using any appropriate method. These include Northern blot analysis, S1 nuclease mapping, the polymerase chain reaction (PCR), reverse transcription in combination with the polymerase chain reaction (RT-PCR), and reverse transcription in combination with the ligase chain reaction (RT-LCR).

In accordance with a further aspect of the invention, there is provided a process for diagnosing or determining a susceptibility to migrane; infections, such as bacterial, fungal, protozoan and viral infections, particularly infection caused by HIV-1 or HIV-2; pain; cancers; anorexia; bulimia; asthma; Parkinson's disease; acute heart failure; hypotension; hypertension; urinary retention; osteoporosis; angina pectoris; myocardial infarction; ulcers; allergies; benign prostatic hypertrophy and psychotic and neurological disorders, including anxiety, schizophrenia, manic depression, delirium, dementia or severe mental retardation; dyskinesias, such as Huntington's disease or Gilles dela Tourett's syndrome, among others. Thus, a mutation in calcitonin receptor indicates a susceptibility to the aforementioned diseases, disorders and conditions and the nucleic acid sequences described above may be employed in an assay for ascertaining such susceptibility. Thus, for example, the assay may be employed to determine a mutation in a human calcitonin receptor protein as herein described, such as a deletion, truncation, insertion, frame shift, etc., with such mutation being indicative of a susceptibility to the aforementioned diseases, disorders and conditions.

The invention provides a process for diagnosing to the aforementioned diseases, disorders and conditions comprising determining from a sample derived from a patient an abnormally decreased or increased level of expression of polynucleotide having the sequence of FIGS. 1A and B (SEQ ID NO: 1). Decreased or increased expression of polynucleotide can be measured using any one of the methods well known in the art for the quantation of polynucleotides, such as, for example, PCR, RT-PCR, RNase protection, Northern blotting and other hybridization methods.

In addition to more conventional gel-electrophoresis and DNA sequencing, mutations can also be detected by in situ analysis.

Polypeptide Assays

The present invention also relates to a diagnostic assays such as quantitative and diagnostic assays for detecting levels of calcitonin receptor protein in cells and tissues, including determination of normal and abnormal levels. Thus, for instance, a diagnostic assay in accordance with the invention for detecting over-expression of calcitonin receptor protein compared to normal control tissue samples may be used to detect the presence of a disease/disorder such as infections, including bacterial, fungal, protozoan and viral infections, particularly infection caused by HIV-1 or HIV-2; pain; cancers; anorexia; bulimia; asthma; Parkinson's disease; acute heart failure; hypotension; hypertension; urinary retention; osteoporosis; angina pectoris; myocardial infarction; ulcers; allergies; benign prostatic hypertrophy and psychotic and neurological disorders, including anxiety, schizophrenia, manic depression, delirium, dementia or severe mental retardation; dyskinesias, such as Huntington's disease or Gilles dela Tourett's syndrome, among others.

Assaying calcitonin receptor protein levels in a biological sample can occur using any art-known method. For example, calcitonin receptor protein expression in tissues can be studied with classical immunohistological methods. (Jalkanen, M., et al., J. Cell. Biol. 101:976-985 (1985); Jalkanen, M., et al., J. Cell. Biol. 105:3087-3096 (1987)).

Other antibody-based methods useful for detecting calcitonin receptor protein gene expression include immunoassays, such as the enzyme linked immunosorbent assay (ELISA) and the radioimmunoassay (RIA).

Suitable labels are known in the art and include enzyme labels, such as glucose oxidase, and radioisotopes, such as iodine (¹²⁵I, ¹²¹I), carbon (¹⁴C), salphee (³⁵S), tritium (³H), indium (¹¹²In), and technetium (^{99m}Tc), and fluorescent labels, such as fluorescein and rhodamine, and biotin.

Antibodies capable of binding to the calcitonin receptor protein antigen may be produced by any art-known method. It will be appreciated that Fab and F(ab′)₂ and other fragments of the antibodies of the present invention may be used according to the methods disclosed herein. Alternatively, calcitonin receptor-binding fragments can be produced through the application of recombinant DNA technology or through synthetic chemistry.

The above-described antibodies may be employed to isolate or to identify clones expressing the polypeptide or purify the polypeptide of the present invention by attachment of the antibody to a solid support for isolation and/or purification by affinity chromatography.

Thus, among others, antibodies against calcitonin receptor may be employed to inhibit to the aforementioned diseases, disorders and conditions.

Calcitonin Receptor Binding Molecules and Assays

Calcitonin receptor could be used to isolate proteins which interact with it and this interaction could be a target for interference. Inhibitors of protein-protein interactions between calcitonin receptor and other factors could lead to the development of pharmaceutical agents for the modulation of calcitonin receptor activity.

Thus, this invention also provides a method for identification of binding molecules to calcitonin receptor. Genes encoding proteins for binding molecules to calcitonin receptor can be identified by numerous methods known to those of skill in the art, for example, ligand panning and FACS sorting. Such methods are described in many laboratory manuals such as, for instance, Coligan et al., Current Protocols in Immunology 1 (Rivett, A. J. Biochem. J. 291, 1-10 (1993)): Chapter 5 (1991).

For example, the yeast two-hybrid system provides methods for detecting the interaction between a first test protein and a second test protein, in vivo, using reconstitution of the activity of a transcriptional activator. The method is disclosed in U.S. Pat. No. 5,283,173; reagents are available from Clontech and Stratagene.

An alternative method is screening of gt11, ZAP (Stratagene) or equivalent cDNA expression libraries with recombinant calcitonin receptor. Recombinant calcitonin receptor protein or fragments thereof are fused to small peptide tags such as FLAG, HSV or GST. The peptide tags can possess convenient phosphorylation sites for a kinase such as heart muscle creatine kinase or they can be biotinylated. Recombinant calcitonin receptor can be phosphorylated with ³²[P] or used unlabeled and detected with streptavidin or antibodies against the tags. gt11cDNA expression libraries are made from cells of interest and are incubated with the recombinant calcitonin receptor, washed and cDNA clones isolated which interact with calcitonin receptor. See, e.g., T. Maniatis et al, infra.

Another method is the screening of a mammalian expression library in which the cDNAs are cloned into a vector between a mammalian promoter and polyadenylation site and transiently transfected in COS or 293 cells followed by detection of the binding protein. See Sims et al., Science 241, 585-589 (1988) and McMahan et al., EMBO J. 10, 2821-2832 (1991). In this manner, pools of cDNAs containing the cDNA encoding the binding protein of interest can be selected and the cDNA of interest can be isolated by further subdivision of each pool followed by cycles of transient transfection, binding and autoradiography. Alternatively, the cDNA of interest can be isolated by transfecting the entire cDNA library into mammalian cells and panning the cells on a dish containing calcitonin receptor bound to the plate. See Seed et al, Proc. Natl. Acad. Sci. USA 84, 3365 (1987) and Aruffo et al., EMBO J. 6, 3313 (1987). If the binding protein is secreted, its cDNA can be obtained by a similar pooling strategy once a binding or neutralizing assay has been established for assaying supernatants from transiently transfected cells. General methods for screening supernatants are disclosed in Wong et al., Science 228, 810-815 (1985).

Another alternative method is immunoaffinity purification well known to those skilled in the art. Yet another alternative method also known to those skilled in the art is screening of peptide libraries for binding partners.

Calcitonin receptor binding partners identified by any of these methods or other methods which would be known to those of ordinary skill in the art as well as those putative binding partners discussed above can be used in the assay method of the invention.

Assays for free calcitonin receptor or binding partner are accomplished by, for example, ELISA or immunoassay using specific antibodies or by incubation of radiolabeled calcitonin receptor with cells or cell membranes followed by centrifugation or filter separation steps. In the presence of test substances which interrupt or inhibit formation of calcitonin receptor/binding partner interaction, an increased amount of free calcitonin receptor or free binding partner will be determined relative to a control lacking the test substance.

Polypeptides of the invention also can be used to assess calcitonin receptor-binding capacity of calcitonin receptor binding molecules in cells or in cell-free preparations.

Calcitonin Receptor Agonists and Antagonists

The G-protein coupled receptor family includes dopamine receptors which bind neuroleptic drugs used for treating psychotic and neurological disorders. Other examples of members of this family include adrenergic, endothelin, cAMP, adenosine, muscarinic, acetylcholine, serotonin, histamine, thrombin, kinin, follicle stimulating hormone, opsins, endothelial differentiation gene-1 receptor, rhodopsins, odorant, cytomegalovirus receptors, etc. Numerous therapeutic agents targeting 7 transmembrane (7 TM) receptors have been successfully introduced onto the market. This indicates that these receptors have an established, proven history as therapeutic targets. G-protein coupled receptors, such as the calcitonin receptor of the present invention, play a role a number of dysfunctions or diseases. Thus, agonists or antagonists of the calcitonin receptor of the present invention could be used therapeutically to prevent, ameliorate or correct dysfunctions or diseases associated with the calcitonin receptor of the present invention.

The calcitonin receptor of the present invention may be employed in a process for screening for compounds which activate (agonists) or inhibit activation (antagonists) of the receptor polypeptide of the present invention.

In general, such screening procedures involve providing appropriate cells which express the receptor polypeptide of the present invention on the surface thereof. Such cells include cells from mammals, yeast, drosophila or E. coli. In particular, a polynucleotide encoding the receptor of the present invention is employed to transfect cells to thereby express the calcitonin receptor. The expressed receptor is then contacted with a test compound to observe binding, stimulation or inhibition of a functional response.

One such screening procedure involves the use of melanophores which are transfected to express the calcitonin receptor of the present invention. Such a screening technique is described in PCT WO 92/01810 published Feb. 6, 1992.

Thus, for example, such assay may be employed for screening for a compound which inhibits activation of the receptor polypeptide of the present invention by contacting the melanophore cells which encode the receptor with both the receptor ligand and a compound to be screened. Inhibition of the signal generated by the ligand indicates that a compound is a potential antagonist for the receptor, i.e., inhibits activation of the receptor.

The screen may be employed for determining a compound which activates the receptor by contacting such cells with compounds to be screened and determining whether such compound generates a signal, i.e., activates the receptor.

Other screening techniques include the use of cells which express the calcitonin receptor (for example, transfected CHO cells) in a system which measures extracellular pH changes caused by receptor activation, for example, as described in Science, 246: 181-296 (October 1989). For example, compounds may be contacted with a cell which expresses the receptor polypeptide of the present invention and a second messenger response, e.g. signal transduction or pH changes, may be measured to determine whether the potential compound activates or inhibits the receptor.

Another method involves screening for compounds which inhibit activation of the receptor polypeptide of the present invention antagonists by determining inhibition of binding of labeled ligand to cells which have the receptor on the surface thereof. Such a method involves transfecting a eukaryotic cell with DNA encoding the calcitonin receptor such that the cell expresses the receptor on its surface and contacting the cell with a compound in the presence of a labeled form of a known ligand. The ligand can be labeled, e.g., by radioactivity. The amount of labeled ligand bound to the receptors is measured, e.g., by measuring radioactivity associated with transfected cells or membrane from these cells. If the compound binds to the receptor as determined by a reduction of labeled ligand which binds to the receptors, the binding of labeled ligand to the receptor is inhibited.

Another method involves screening for calcitonin receptor inhibitors by determining inhibition or stimulation of calcitonin receptor-mediated cAMP and/or adenylate cyclase accumulation. Such a method involves transfecting a eukaryotic cell with calcitonin receptor to express the receptor on the cell surface. The cell is then exposed to potential antagonists in the presence of calcitonin receptor. The amount of cAMP accumulation is then measured. If the potential antagonist binds the receptor, and thus inhibits calcitonin receptor binding, the levels of calcitonin receptor-mediated cAMP, or adenylate cyclase, activity will be reduced or increased.

The present invention also provides a method for determining whether a ligand not known to be capable of binding to a calcitonin receptor can bind to such receptor which comprises contacting a mammalian cell which expresses a calcitonin receptor with the ligand under conditions permitting binding of ligands to the calcitonin receptor, detecting the presence of a ligand which binds to the receptor and thereby determining whether the ligand binds to the calcitonin receptor. The systems hereinabove described for determining agonists and/or antagonists may also be employed for determining ligands which bind to the receptor.

Examples of potential calcitonin receptor receptor antagonists are an antibody, or in some cases an oligonucleotide, which binds to the receptor but does not elicit a second messenger response such that the activity of the receptor is prevented.

Potential antagonists also include proteins which are closely related to the ligand of the calcitonin receptor, i.e. a fragment of the ligand, which have lost biological function and when binding to the calcitonin receptor, elicit no response.

A potential antagonist also includes an antisense construct prepared through the use of antisense technology. Antisense technology can be used to control gene expression through triple-helix formation or antisense DNA or RNA, both of which methods are based on binding of a polynucleotide to DNA or RNA. For example, the 5′ coding portion of the polynucleotide sequence, which encodes for the polypeptides of the present invention, is used to design an antisense RNA oligonucleotide of from about 10 to 40 base pairs in length. A DNA oligonucleotide is designed to be complementary to a region of the gene involved in transcription (triple helix-see Lee et al., Nucl. Acids Res., 6:3073 (1979); Cooney et al, Science, 241:456 (1988); and Dervan et al., Science, 251: 1360 (1991)), thereby preventing transcription and the production of calcitonin receptor. The antisense RNA oligonucleotide hybridizes to the mRNA in vivo and blocks translation of the mRNA molecule into the calcitonin receptor (antisense-Okano, J. Neurochem., 56:560 (1991); Oligodeoxynucleotides as Antisense Inhibitors of Gene Expression, CRC Press, Boca Raton, Fla. (1988)). The oligonucleotides described above can also be delivered to cells such that the antisense RNA or DNA may be expressed in vivo to inhibit production of the calcitonin receptor.

Another potential antagonist is a small molecule which binds to the calcitonin receptor, making it inaccessible to ligands such that normal biological activity is prevented. Examples of small molecules include but are not limited to small peptides or peptide-like molecules.

Potential antagonists also include a soluble form of a calcitonin receptor, e.g. a fragment of the receptor, which binds to the ligand and prevents the ligand from interacting with membrane bound calcitonin receptors.

Calcitonin receptor are ubiquitous in the mammalian host and are responsible for many biological functions, including many pathologies. Accordingly, it is desirous to find compounds and drugs which stimulate the calcitonin receptor on the one hand and which can inhibit the function of a calcitonin receptor on the other hand.

In general, agonists or antagonists for calcitonin receptor are employed for therapeutic and prophylactic purposes for such diseases or disorders as infections, such as bacterial, fungal, protozoan and viral infections, particularly infection caused by HIV-1 or HIV-2; pain; cancers; anorexia; bulimia; asthma; Parkinson's disease; acute heart failure; hypotension; hypertension; urinary retention; osteoporosis; angina pectoris; myocardial infarction; ulcers; allergies; benign prostatic hypertrophy and psychotic and neurological disorders, including anxiety, schizophrenia, manic depression, delirium, dementia or severe mental retardation; dyskinesias, such as Huntington's disease or Gilles dela Tourett's syndrome, among others.

Modes of Administration

It will be appreciated that conditions caused by a decrease in the standard or normal level of calcitonin receptor activity in an individual, can be treated by administration of agonists of the calcitonin receptor protein. The invention further provides a method of treating an individual in need of an increased level of calcitonin receptor activity comprising administering to such an individual a pharmaceutical composition comprising an effective amount of an agonist of the calcitonin receptor of the invention, effective to increase the calcitonin receptor activity level in such an individual.

As a general proposition, the total pharmaceutically effective amount of calcitonin receptor agonist or antagonist administered parenterally per dose will be in the range of about 1 μg/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day for the hormone. If given continuously, the calcitonin receptor polypeptide is typically administered at a dose rate of about 1 μg/kg/hour to about 50 μg/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Pharmaceutical compositions containing the calcitonin receptor agonist or antagonist may be administered orally, rectally, parenterally, intracistemally, intravaginally, intraperitoneally, topically (as by powders, ointments, drops or transdermal patch), bucally, or as an oral or nasal spray. By “pharmaceutically acceptable carrier” is meant a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion.

Chromosome Assays

The nucleic acid molecules of the present invention are also valuable for chromosome identification. The sequence is specifically targeted to and can hybridize with a particular location on an individual human chromosome. The mapping of DNAs to chromosomes according to the present invention is an important first step in correlating those sequences with genes associated with disease.

In certain preferred embodiments in this regard, the cDNA herein disclosed is used to clone genomic DNA of a calcitonin receptor protein gene. This can be accomplished using a variety of well known techniques and libraries, which generally are available commercially. The genomic DNA then is used for in situ chromosome mapping using well known techniques for this purpose. Typically, in accordance with routine procedures for chromosome mapping, some trial and error may be necessary to identify a genomic probe that gives a good in situ hybridization signal.

In addition, in some cases, sequences can be mapped to chromosomes by preparing PCR primers preferably 15-25 bp) from the cDNA. Computer analysis of the 3′ untranslated region of the gene is used to rapidly select primers that do not span more than one exon in the genomic DNA, thus complicating the amplification process. These primers are then used for PCR screening of somatic cell hybrids containing individual human chromosomes.

Fluorescence in situ hybridization (“FISH”) of a cDNA clone to a metaphase chromosomal spread can be used to provide a precise chromosomal location in one step. This technique can be used with probes from the cDNA as short as 50 or 60 bp. For a review of this technique, see Verma et al., Human Chromosomes: A Manual Of Basic Techniques, Pergamon Press, New York (1988).

Once a sequence has been mapped to a precise chromosomal location, the physical position of the sequence on the chromosome can be correlated with genetic map data. Such data are found, for example, in V. McKusick, Mendelian Inheritance In Man, available on-line through Johns Hopkins University, Welch Medical Library. The relationship between genes and diseases that have been mapped to the same chromosomal region are then identified through linkage analysis (coinheritance of physically adjacent genes).

Next, it is necessary to determine the differences in the cDNA or genomic sequence between affected and unaffected individuals. If a mutation is observed in some or all of the affected individuals but not in any normal individuals, then the mutation is likely to be the causative agent of the disease.

Having generally described the invention, the same will be more readily understood by reference to the following examples, which are provided by way of illustration and are not intended as limiting.

### EXAMPLES

### Example 1

Expression and Purification of Calcitonin Receptor in E. coli

The DNA sequence encoding the calcitonin receptor protein in the deposited cDNA clone is amplified using PCR oligonucleotide primers specific to the amino terminal sequences of the calcitonin receptor protein and to vector sequences 3′ to the gene. Additional nucleotides containing restriction sites to facilitate cloning are added to the 5′ and 3′ sequences respectively.

The 5′ oligonucleotide primer has the sequence 5′ GCCAGGTCGACCACATGACGAGCTTCGCT 3′(SEQ ID NO:3) containing the underlined Sal I restriction site, which encodes 15 nucleotides of the calcitonin receptor protein coding sequence in SEQ ID NO: 1.

The 3 ′ primer has the sequence 5′ CGGCTCTAGAGTTAATGCACGAAGTTAAAGAAG 3′ (SEQ ID NO:4) containing the underlined Xba I restriction site followed by nucleotides complementary to nucleotides immediately after the calcitonin receptor protein coding sequence in SEQ ID NO:1.

The restriction sites are convenient to restriction enzyme sites in the bacterial expression vector pD10 (pQE9), which are used for bacterial expression in these examples. (Qiagen, Inc. 9259 Eton Avenue, Chatsworth, Calif., 9131 1). [pD10]pQE9 encodes ampicillin antibiotic resistance (“Amp”) and contains a bacterial origin of replication (“ori”), an IPTG inducible promoter, a ribosome binding site (“RBS”), a 6-His tag and restriction enzyme sites.

The amplified calcitonin receptor DNA and the vector pQE9 both are digested with SalI and XbaI and the digested DNAs are then ligated together. Insertion of the calcitonin receptor protein DNA into the restricted pQE9 vector places the calcitonin receptor protein coding region downstream of and operably linked to the vector's IPTG-inducible promoter and in-frame with an initiating AUG appropriately positioned for translation of calcitonin receptor protein.

The ligation mixture is transformed into competent E. coli cells using standard procedures. Such procedures are described in Sambrook et al., Molecular Cloning: a Laboratory Manual, 2nd Ed.; Cold Spring Harbor Laboratory Press, Cold Spring Harbor, N.Y. (1989). E. coli strain M15/rep4, containing multiple copies of the plasmid pREP4, which expresses lac repressor and confers kanamycin resistance (“Kan”), is used in carrying out the illustrative example described herein. This strain, which is only one of many that are suitable for expressing calcitonin receptor protein, is available commercially from Qiagen.

Transformants are identified by their ability to grow on LB plates in the presence of ampicillin and kanamycin. Plasmid DNA is isolated from resistant colonies and the identity of the cloned DNA confirmed by restriction analysis.

Clones containing the desired constructs are grown overnight (“O/N”) in liquid culture in LB media supplemented with both ampicillin (100 μg/ml) and kanamycin (25 μg/ml).

The O/N culture is used to inoculate a large culture, at a dilution of approximately 1:100 to 1:250. The cells are grown to an optical density at 600 nm (“OD600”) of between 0.4 and 0.6. Isopropyl-B-D-thiogalactopyranoside (“IPTG”) is then added to a final concentration of 1 mM to induce transcription from lac repressor sensitive promoters, by inactivating the lacI repressor. Cells subsequently are incubated further for 3 to 4 hours. Cells then are harvested by centrifugation and disrupted, by standard methods. Inclusion bodies are purified from the disrupted cells using routine collection techniques, and protein is solubilized from the inclusion bodies into 8M urea. The 8M urea solution containing the solubilized protein is passed over a PD-10 column in 2X phosphate-buffered saline (“PBS”), thereby removing the urea, exchanging the buffer and refolding the protein. The protein is purified by a further step of chromatography to remove endotoxin. Then, it is sterile filtered. The sterile filtered protein preparation is stored in 2X PBS at a concentration of 95 μ/ml.

### Example 2

Cloning and Expression of Calcitonin Receptor protein in a Baculovirus Expression System

The cDNA sequence encoding the full length calcitonin receptor protein in the deposited clone is amplified using PCR oligonucleotide primers corresponding to the 5′ and 3′ sequences of the gene:

The 5′ primer has the sequence 5′GTCATCTAGAAGTCCGCCATCATGACGAGCAGCTTCGCTGTGCTC 3′ (SEQ ID NO:5) containing the underlined Xba I restriction enzyme site followed by 21 bases of the sequence of calcitonin receptor protein in SEQ ID NO: 1. Inserted into an expression vector, as described below, the 5′ end of the amplified fragment encoding calcitonin receptor provides an efficient signal peptide. An efficient signal for initiation of translation in eukaryotic cells, as described by Kozak, M., J. Mol. Biol. 196:947-950 (1987) is appropriately located in the vector portion of the construct.

The 3′ primer has the sequence 5′ CGGCTCTAGAGTTAATGCACGAAGTTAAAGAAG 3′ (SEQ ID NO:6) containing the underlined Xba I restriction site followed by nucleotides complementary to the last 19 nucleotides of the calcitonin receptor coding sequence set out in SEQ ID NO: 1.

The amplified fragment is isolated from a 1% agarose gel using a commercially available kit (“Geneclean,” BIO 101 Inc., La Jolla, Calif.). The fragment then is digested with XbaI and again is purified on a 1% agarose gel. This fragment is designated herein F2.

The vector pA2-GP is used to express the calcitonin receptor protein in the baculovirus expression system, using standard methods, as described in Summers et al., A Manual of Methods for Baculovirus Vectors and Insect Cell Culture Procedures, Texas Agricultural Experimental Station Bulletin No. 1555 (1987). This expression vector contains the strong polyhedrin promoter of the Autographa californica nuclear polyhedrosis virus (AcMNPV) followed by convenient restriction sites. The signal peptide of AcMNPV gp67, including the N-terminal methionine, is located just upstream of a BamHI site. The polyadenylation site of the simian virus 40 (“SV40”) is used for efficient polyadenylation. For an easy selection of recombinant virus the beta-galactosidase gene from E. coli is inserted in the same orientation as the polyhedrin promoter and is followed by the polyadenylation signal of the polyhedrin gene. The polyhedrin sequences are flanked at both sides by viral sequences for cell-mediated homologous recombination with wild-type viral DNA to generate viable virus that express the cloned polynucleotide.

Many other baculovirus vectors could be used in place of pA2-GP, such as pAc373, pVL941 and pAcIM1 provided, as those of skill readily will appreciate, that construction provides appropriately located signals for transcription, translation, trafficking and the like, such as an in-frame AUG and a signal peptide, as required. Such vectors are described in Luckow et al., Virology 170: 31-39, among others.

The plasmid is digested with the restriction enzyme XbaI and then is dephosphorylated using calf intestinal phosphatase, using routine procedures known in the art. The DNA is then isolated from a 1% agarose gel using a commercially available kit (“Geneclean” BIO 101 Inc., La Jolla, Calif.). This vector DNA is designated herein “V”.

Fragment F2 and the dephosphorylated plasmid V2 are ligated together with T4 DNA ligase. E. coli HB101 cells are transformed with ligation mix and spread on culture plates. Bacteria are identified that contain the plasmid with the human calcitonin receptor gene by digesting DNA from individual colonies using XbaI and then analyzing the digestion product by gel electrophoresis. The sequence of the cloned fragment is confirmed by DNA sequencing. This plasmid is designated herein pBaccalcitonin receptor.

5 μg of the plasmid pBaccalcitonin receptor is co-transfected with 1.0 μg of a commercially available linearized baculovirus DNA (“BaculoGold™ baculovirus DNA”, Pharmingen, San Diego, Calif.), using the lipofection method described by Felgner et al., Proc. Natl. Acad. Sci. USA 84: 7413-7417 (1987). 1 μg of BaculoGold™ virus DNA and 5 μg of the plasmid pBaccalcitonin receptor are mixed in a sterile well of a microtiter plate containing 50 μl of serum-free Grace's medium (Life Technologies Inc., Gaithersburg, Md.). Afterwards 10 μl Lipofectin plus 90 μl Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is rocked back and forth to mix the newly added solution. The plate is then incubated for 5 hours at 27° C. After 5 hours the transfection solution is removed from the plate and 1 ml of Grace's insect medium supplemented with 10% fetal calf serum is added. The plate is put back into an incubator and cultivation is continued at 27° C. for four days.

After four days the supernatant is collected and a plaque assay is performed, as described by Summers and Smith, cited above. An agarose gel with “Blue Gal” (Life Technologies Inc., Gaithersburg) is used to allow easy identification and isolation of gal-expressing clones, which produce blue-stained plaques. (A detailed description of a “plaque assay” of this type can also be found in the user's guide for insect cell culture and baculovirology distributed by Life Technologies Inc., Gaithersburg, page 9-10).

Four days after serial dilution, the virus is added to the cells. After appropriate incubation, blue stained plaques are picked with the tip of an Eppendorf pipette. The agar containing the recombinant viruses is then resuspended in an Eppendorf tube containing 200 μl of Grace's medium. The agar is removed by a brief centrifugation and the supernatant containing the recombinant baculovirus is used to infect Sf9 cells seeded in 35 mm dishes. Four days later the supernatants of these culture dishes are harvested and then they are stored at 4° C. A clone containing properly inserted hESSB I, II and III is identified by DNA analysis including restriction mapping and sequencing. This is designated herein as V-calictonin receptor.

Sf9 cells are grown in Grace's medium supplemented with 10% heat-inactivated FBS. The cells are infected with the recombinant baculovirus V-calcitonin receptor at a multiplicity of infection (“MOI”) of about 2 (about 1 to about 3). Six hours later the medium is removed and is replaced with SF900 II medium minus methionine and cysteine (available from Life Technologies Inc., Gaithersburg). 42 hours later, 5 μCi of ³⁵S-methionine and 5 μCi ³⁵S-cysteine (available from Amersham) are added. The cells are further incubated for 16 hours and then they are harvested by centrifugation, lysed and the labeled proteins are visualized by SDS-PAGE and autoradiography.

### Example 3

Cloning and Expression in Mammalian Cells

Most of the vectors used for the transient expression of the calcitonin receptor protein gene sequence in mammalian cells should carry the SV40 origin of replication. This allows the replication of the vector to high copy numbers in cells (e.g., COS cells) which express the T antigen required for the initiation of viral DNA synthesis. Any other mammalian cell line can also be utilized for this purpose.

A typical mammalian expression vector contains the promoter element, which mediates the initiation of transcription of mRNA, the protein coding sequence, and signals required for the termination of trancription and polyadenylation of the transcript. Additional elements include enhancers, Kozak sequences and intervening sequences flanked by donor and acceptor sites for RNA splicing. Highly efficient transcription can be achieved with the early and late promoters from SV40, the long terminal repeats (LTRs) from Retroviruses, e.g., RSV, HTLVI, HIVI and the early promoter of the cytomegalovirus (CMV). However, cellular signals can also be used (e.g., human actin promoter). Suitable expression vectors for use in practicing the present invention include, for example, vectors such as pSVL and pMSG (Pharmacia, Uppsala, Sweden), pRSVcat (ATCC 37152), pSV2dhfr (ATCC 37146) and pBC12MI (ATCC 67109). Mammalian host cells that could be used include, human Hela, 283, H9 and Jurkart cells, mouse NIH3T3 and C127 cells, Cos 1, Cos 7 and CV1, African green monkey cells, quail QC1-3 cells, mouse L cells and Chinese hamster ovary cells.

Alternatively, the gene can be expressed in stable cell lines that contain the gene integrated into a chromosome. The co-transfection with a selectable marker such as dhfr, gpt, neomycin, hygromycin allows the identification and isolation of the transfected cells.

The transfected gene can also be amplified to express large amounts of the encoded protein. The DHFR (dihydrofolate reductase) is a useful marker to develop cell lines that carry several hundred or even several thousand copies of the gene of interest. Another useful selection marker is the enzyme glutamine synthase (GS) (Murphy et al., Biochem J. 227:277-279 (1991); Bebbington et al., Bio/Technology 10:169-175 (1992)). Using these markers, the mammalian cells are grown in selective medium and the cells with the highest resistance are selected. These cell lines contain the amplified gene(s) integrated into a chromosome. Chinese hamster ovary (CHO) cells are often used for the production of proteins.

The expression vectors pC1 and pC4 contain the strong promoter (LTR) of the Rous Sarcoma Virus (Cullen et al., Molecular and Cellular Biology, 438-447 (March, 1985)) plus a fragment of the CMV-enhancer (Boshart et al., Cell 41:521-530 (1985)). Multiple cloning sites, e.g., with the restriction enzyme cleavage sites BamHI, XbaI and Asp718, facilitate the cloning of the gene of interest. The vectors contain in addition the 3′ intron, the polyadenylation and termination signal of the rat preproinsulin gene.

### Example 3(a)

Cloning and Expression in COS Cells

The expression plasmid, pCalcitonin ReceptorHA, is made by cloning a cDNA encoding calcitonin receptor into the expression vector pcDNAI/Amp (which can be obtained from Invitrogen, Inc.).

The expression vector pcDNAI/amp contains: (1) an E. coli origin of replication effective for propagation in E. coli and other prokaryotic cells; (2) an ampicillin resistance gene for selection of plasmid-containing prokaryotic cells; (3) an SV40 origin of replication for propagation in eukaryotic cells; (4) a CMV promoter, a polylinker, an SV40 intron, and a polyadenylation signal arranged so that a cDNA conveniently can be placed under expression control of the CMV promoter and operably linked to the SV40 intron and the polyadenylation signal by means of restriction sites in the polylinker.

A DNA fragment encoding the calcitonin receptor protein and an HA tag fused in frame to its 3′ end is cloned into the polylinker region of the vector so that recombinant protein expression is directed by the CMV promoter. The HA tag corresponds to an epitope derived from the influenza hemagglutinin protein described by Wilson et al., Cell 37.: 767 (1984). The fusion of the HA tag to the target protein allows easy detection of the recombinant protein with an antibody that recognizes the HA epitope.

The plasmid construction strategy is as follows. The calcitonin receptor cDNA of the deposited clone is amplified using primers that contain convenient restriction sites, much as described above regarding the construction of expression vectors for expression of calcitonin receptor in E. coli. To facilitate detection, purification and characterization of the expressed calcitonin receptor, one of the primers contains a hemagglutinin tag (“HA tag”) as described above.

Suitable primers include the following, which are used in this example. The 5′ primer, containing the underlined Xba I site, an AUG start codon and 21 bases of the 5′ coding region has the following sequence:

5 ′GTCATCTAGAAGTCCGCCATCATGACGAGCTTCGCTG TGCTC 3′ (SEQ ID NO:7).

The 3′ primer, containing the underlined Xba I site, a stop codon, 9 codons thereafter forming the hemagglutinin HA tag, and 19 bases of 3′ coding sequence (at the 3′ end) has the following sequence:

5′ CGGCTCTAGAGTTAAGCGTAGTCTGGGACGTCGTATG GGTAATGCACGAAGGTTAAAGAAG 3′ (SEQ ID NO:8).

The following 3′ primer, containing the underlined Xba I site, a stop codon, and 19 bases of 3′ coding sequence (at the 3′ end) can be used to express a protein lacking the HA tag. This primer has the following sequence:

5′ CGGCTCTAGAGTTAATGCACGAAGTTAAAGAAG 3′ (SEQ ID NO:9).

The PCR amplified DNA fragment and the vector, pcDNAI/Amp, are digested with HindIII and XhoI and then ligated. The ligation mixture is transformed into E. coli strain SURE (available from Stratagene Cloning Systems, 11099 North Torrey Pines Road, La Jolla, Calif. 92037), and the transformed culture is plated on ampicillin media plates which then are incubated to allow growth of ampicillin resistant colonies. Plasmid DNA is isolated from resistant colonies and examined by restriction analysis and gel sizing for the presence of the calcitonin receptor-encoding fragment.

For expression of recombinant calcitonin receptor, COS cells are transfected with an expression vector, as described above, using DEAE-DEXTRAN, as described, for instance, in Sambrook et al., Molecular Cloning: a Laboratory Manual, Cold Spring Laboratory Press, Cold Spring Harbor, New York (1989). Cells are incubated under conditions for expression of calcitonin receptor by the vector.

Expression of the calcitonin receptor HA fusion protein is detected by radiolabelling and immunoprecipitation, using methods described in, for example Harlow et al., Antibodies: A Laboratory Manual, 2nd Ed.; Cold Spring Harbor Laboratory Press, Cold Spring Harbor, New York (1988). To this end, two days after transfection, the cells are labeled by incubation in media containing ³⁵S-cysteine for 8 hours. The cells and the media are collected, and the cells are washed and the lysed with detergent-containing RIPA buffer: 150 mM NaCl, 1% NP-40, 0.1% SDS, 1% NP-40, 0.5% DOC, 50 mM TRIS, pH 7.5, as described by Wilson et al. cited above. Proteins are precipitated from the cell lysate and from the culture media using an HA-specific monoclonal antibody. The precipitated proteins then are analyzed by SDS-PAGE gels and autoradiography. An expression product of the expected size is seen in the cell lysate, which is not seen in negative controls.

### Example 3(b)

Cloning and Expression in CHO Cells

The vector pC4 is used for the expression of calcitonin receptor protein. Plasmid pC4 is a derivative of the plasmid pSV2-dhfr [ATCC Accession No. 37146]. Both plasmids contain the mouse DHFR gene under control of the SV40 early promoter. Chinese hamster ovary- or other cells lacking dihydrofolate activity that are transfected with these plasmids can be selected by growing the cells in a selective medium (alpha minus MEM, Life Technologies) supplemented with the chemotherapeutic agent methotrexate. The amplification of the DHFR genes in cells resistant to methotrexate (MTX) has been well documented (see, e.g., Alt, F. W., Kellems, R. M., Bertino, J. R., and Schimke, R. T., 1978, J. Biol. Chem. 253:1357-1370, Hamlin, J. L. and Ma, C. 1990, Biochem. et Biophys. Acta, 1097:107-143, Page, M. J. and Sydenham, M. A. 1991, Biotechnology Vol. 9:64-68). Cells grown in increasing concentrations of MTX develop resistance to the drug by overproducing the target enzyme, DHFR, as a result of amplification of the DHFR gene. If a second gene is linked to the DHFR gene it is usually co-amplified and over-expressed. It is state of the art to develop cell lines carrying more than 1,000 copies of the genes. Subsequently, when the methotrexate is withdrawn, cell lines contain the amplified gene integrated into the chromosome(s).

Plasmid pC4 contains for expressing the gene of interest the strong promoter of the long terminal repeat (LTR) of the Rous Sarcoma Virus (Cullen, et al., Molecular and Cellular Biology, March 1985:438447) plus a fragment isolated from the enhancer of the immediate early gene of human cytomegalovirus (CMV) (Boshart et al, Cell 41:521-530 (1985)). Downstream of the promoter are Bam HI, XbaI, and Asp718 restriction enzyme cleavage sites that allow integration of the genes. Behind these cloning sites the plasmid contains the 3′ intron and polyadenylation site of the rat preproinsulin gene. Other high efficiency promoters can also be used for the expression, e.g., the human β-actin promoter, the SV40 early or late promoters or the long terminal repeats from other retroviruses, e.g., HIV and HTLVI. Clontech's Tet-Off and Tet-On gene expression systems and similar systems can be used to express the Calcitonin Receptor in a regulated way in mammalian cells (Gossen, M., & Bujard, H. 1992, Proc. Natl. Acad Sci. USA 89:5547-5551). For the polyadenylation of the mRNA other signals, e.g., from the human growth hormone or globin genes can be used as well. Stable cell lines carrying a gene of interest integrated into the chromosomes can also be selected upon co-transfection with a selectable marker such as gpt, G418 or hygromycin. It is advantageous to use more than one selectable marker in the beginning, e.g., G418 plus methotrexate.

Stable cell lines carrying a gene of interest integrated into the chromosomes can also be selected upon co-transfection with a selectable marker such as gpt, G418 or hygromycin. It is advantageous to use more than one selectable marker in the beginning, e.g., G418 plus methotrexate.

The plasmid pC4 is digested with the restriction enzyme Xba I and then dephosphorylated using calf intestinal phosphates by procedures known in the art. The vector is then isolated from a 1% agarose gel.

The DNA sequence encoding calcitonin receptor, ATCC No. 97730, is amplified using PCR oligonucleotide primers corresponding to the 5′ and 3′ sequences of the gene:

The 5′ primer has the sequence 5′ GTCATCTAGAAGTCCGCCATCATGACGAGCGGCGCTGTGCTC 3′(SEQ ID NO: 10) containing the underlined Xba I restriction enzyme site followed by 21 bases of the sequence of calcitonin receptor of SEQ ID NO: 1. Inserted into an expression vector, as described below, the 5′ end of the amplified fragment encoding human calcitonin receptor provides an efficient signal peptide. An efficient signal for initiation of translation in eukaryotic cells, as described by Kozak, M., J. Mol. Biol. 196:947-950 (1987) is appropriately located in the vector portion of the construct.

The 3′ primer, containing the underlined Xba I site, a stop codon, 9 codons thereafter forming the hemagglutinin HA tag, and 19 bases complementary to the 3′ coding sequence (at the 3′ end) has the following sequence:

5′ CGGCTCTAGAGTTAAGCGTAGTCTGGGACGTCGTATGGGTAATGCA CGAAGGTTAAAGAAG 3′ (SEQ ID NO:11).

The following 3′ primer, containing the underlined Xba I site, a stop codon, and 19 bases of 3′ coding sequence (at the 3′ end) can be used to express a protein lacking the HA tag. This primer has the following sequence:

5′ CGGCTCTAGAGTTAATGCACGAAGTTAAAGAAG 3′ (SEQ ID NO: 12).

The amplified fragment is digested with the endonuclease Xba I and then purified again on a 1% agarose gel. The isolated fragment and the dephosphorylated vector are then ligated with T4 DNA ligase. E. coli HB101 or XL-1 Blue cells are then transformed and bacteria are identified that contain the fragment inserted into plasmid pC4 using, for instance, restriction enzyme analysis.

Chinese hamster ovary cells lacking an active DHFR gene are used for transfection. 5 μg of the expression plasmid pC4 is cotransfected with 0.5 μg of the plasmid pSV2-neo using lipofectin (Felgner et al., supra). The plasmid pSV2neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of metothrexate plus 1 mg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 μM, 2 μM, 5 μM, 10 mM, 20 mM). The same procedure is repeated until clones are obtained which grow at a concentration of 100-200 μM. Expression of the desired gene product is analyzed, for instance, by SDS-PAGE and Western blot or by reverse phase HPLC analysis.

### Example 4

Tissue Distribution of Calcitonin Receptor Protein Expression

Northern blot analysis is carried out to examine calcitonin receptor gene expression in human tissues, using methods described by, among others, Sambrook et al., cited above. A cDNA probe containing the entire nucleotide sequence of the calcitonin receptor protein (SEQ ID NO:1) is labeled with 32P using the rediprime™ DNA labeling system (Amersham Life Science), according to manufacturer's instructions. After labelling, the probe is purified using a CHROMA SPIN-100™ column (Clontech Laboratories, Inc.), according to manufacturer's protocol number PT1200-1. The purified labelled probe is then used to examine various human tissues for calcitonin receptor mRNA.

Multiple Tissue Northern (MTN) blots containing various human tissues (H) or human immune system tissues (IM) are obtained from Clontech and are examined with labelled probe using ExpressHyb™ hybridization solution (Clontech) according to manufacturer's protocol number PT1190-1. Following hybridization and washing, the blots are mounted and exposed to film at −70° C. overnight, and films developed according to standard procedures.

Similarly, Northern blots of fetal tissue or cancerous tissues are also examined with labelled probe using ExpressHyb™ hybridization solution (Clontech) according to manufacturer's protocol number PT1190-1. Following hybridization and washing, the blots are mounted and exposed to film at −70° C. overnight, and films developed according to standard procedures.

It will be clear that the invention may be practiced otherwise than as particularly described in the foregoing description and examples.

Numerous modifications and variations of the present invention are possible in light of the above teachings and, therefore, are within the scope of the appended claims.

The entire disclosure of all publications (including patents, patent applications, journal articles, laboratory manuals, books, or other documents) cited herein are hereby incorporated by reference.

What is claimed is:

1. An isolated nucleic acid molecule comprising a polynucleotide having a nucleotide sequence at least 95% identical to a sequence selected from the group consisting of:
(a) a nucleotide sequence encoding the calcitonin receptor polypeptide having the amino acid sequence from about 1 to about 568 in SEQ ID NO:2;
(b) a nucleotide sequence encoding the calcitonin receptor polypeptide having the amino acid sequence from about 2 to about 568 in SEQ ID NO:2;
(c) a nucleotide sequence encoding the calcitonin receptor polypeptide having the amino acid sequence encoded by the cDNA clone contained in ATCC Deposit No. 97730;
(d) a nucleotide sequence encoding a polypeptide comprising the calcitonin receptor extracellular regions selected from the group consisting of: amino acid residues from about 1 to about 18, amino acid residues from about 148 to about 159, amino acid residues from about 188 to about 209, and amino acid residues from about 231 to about 236 in SEQ ID NO:2;
(e) a nucleotide sequence encoding a polypeptide comprising the calcitonin receptor intracellular regions selected from the group consisting of:amino acid residues from about 50 to about 59, amino acid residues from about 110 to about 125, amino acid residues from about 188 to about 209, and amino acid residues from about 259 to about 560 in SEQ ID NO:2;
(f) a nucleotide sequence encoding a polypeptide comprising the calcitonin receptor transmembrane domain 1 (amino acid residues from about 20 to about 49 in SEQ ID NO:2);
(g) a nucleotide sequence encoding a polypeptide comprising the calcitonin receptor transmembrane domain 2 (amino acid residues from about 60 to about 80 in SEQ ID NO:2);
(h) a nucleotide sequence encoding a polypeptide comprising the calcitonin receptor transmembrane domain 3 (amino acid residues from about 82 to about 109 in SEQ ID NO:2);
(i) a nucleotide sequence encoding a polypeptide comprising the calcitonin receptor transmembrane domain 4 (amino acid residues from about 126 to about 147 in SEQ ID NO:2);
(j) a nucleotide sequence encoding the calcitonin receptor transmembrane domain 5 (amino acid residues from about 160 to about 187 in SEQ ID NO:2);
(k) a nucleotide sequence encoding a polypeptide comprising the calcitonin receptor transmembrane domain 6 (amino acid residues from about 209 to about 230 in SEQ ID NO:2);
(l) a nucleotide sequence encoding a polypeptide comprising the calcitonin receptor transmembrane domain 7 (amino acid residues from about 237 to about 258 in SEQ ID NO:2); and
(m) a nucleotide sequence complementary to any of the nucleotide sequences in (a), (b), (c), (d), (e), (f), (g), (h), (i), (j), (k) or (l).

2. An isolated nucleic acid molecule comprising a polynucleotide which encodes the amino acid sequence of an epitope-bearing portion of an calcitonin receptor polypeptide having an amino acid sequence in (a), (b), (c), (d), (e), (f), (g), (h), (i), (j), (k) or (l) of claim 1.

3. The isolated nucleic acid molecule of claim 2, which encodes an epitope-bearing portion of any of the calcitonin receptor polypeptide selected from the group consisting of: a polypeptide comprising amino acid residues from about 10 to about 20 in SEQ ID NO:2; a polypeptide comprising amino acid residues from about 49 to about 60 in SEQ ID NO:2; a polypeptide comprising amino acid residues from about 113 to about 123 in SEQ ID NO:2; a polypeptide comprising amino acid residues from about 145 to about 154 in SEQ ID NO:2; a polypeptide comprising amino acid residues from about 189 to about 209 in SEQ ID NO:2; and a polypeptide comprising amino acid residues from about 259 to about 560 in SEQ ID NO:2.

4. An isolated nucleic acid molecule, comprising a polynucleotide having a sequence slected from the group consisting of:
(a) the nucleotide sequence of a fragment of the sequence shown in SEQ ID NO: 1, wherein said fragment comprises at least 50 contiguous nucleotides of SEQ ID NO:1, provided that said isolated nucleic acid molecule is not HCEPR64R (SEQ ID NO:13), HCETE57R (SEQ ID NO:14), R60111 (SEQ ID NO:15), R60049 (SEQ ID NO:16), AA326354 (SEQ ID NO:17), W52864 (SEQ ID NO:18), AA037392 (SEQ ID NO: 19), T06233 (SEQ ID NO:20), AA477598 (SEQ ID NO:21), F13341 (SEQ ID NO:22), AA477470 (SEQ ID NO:23), T77092 (SEQ ID NO:24), H55526 (SEQ ID NO:25), AA349458 (SEQ ID NO:26) or any subfragment thereof; and
(b) a nucleotide sequence complementary to a nucleotide sequence in (a).

5. A method for making a recombinant vector comprising inserting an isolated nucleic acid molecule of claim 1 into a vector.

6. A recombinant vector produced by the method of claim 5.

7. A method of making a recombinant host cell comprising introducing the recombinant vector of claim 6 into a host cell.

8. A recombinant host cell produced by the method of claim 7.

9. A recombinant method for producing any of the calcitonin receptor polypeptide, comprising culturing the recombinant host cell of claim 8 under conditions such that said polypeptide is expressed and recovering said polypeptide.

10. An isolated calcitonin receptor polypeptide having an amino acid sequence at least 95% identical to a sequence selected from the group consisting of:
(a) the amino acid sequence of the calcitonin receptor polypeptide comprising the amino acid sequence from about 1 to about 568 in SEQ ID NO:2;
(b) the amino acid sequence of the calcitonin receptor polypeptide comprising the amino acid sequence from about 2 to about 568 in SEQ ID NO:2;
(c) the amino acid sequence of the calcitonin receptor polypeptide having the amino acid sequence encoded by the eDNA clone contained in ATCC Deposit No. 97730;
(d) the amino acid sequence of the calcitonin receptor extracellular regions selected from the group consisting of: amino acid residues from about 1 to about 18, amino acid residues from about 148 to about 159, amino acid residues from about 188 to about 209, and amino acid residues from about 231 to about 236 in SEQ ID NO:2;
(e) the amino acid sequence of the calcitonin receptor intracellular regions selected from the group consisting of:amino acid residues from about 50 to about 59, amino acid residues from about 110 to about 125, amino acid residues from about 188 to about 209, and amino acid residues from about 259 to about 560 in SEQ ID NO:2;
(f) the amino acid sequence of the calcitonin receptor transmembrane domain 1 (amino acid residues from about 20 to about 49 in SEQ ID NO:2);
(g) the amino acid sequence of the calcitonin receptor transmembrane domain 2 (amino acid residues from about 60 to about 80 in SEQ ID NO:2);
(h) the amino acid sequence of the calcitonin receptor transmembrane domain 3 (amino acid residues from about 82 to about 109 in SEQ ID NO:2);
(i) the amino acid sequence of the calcitonin receptor transmembrane domain 4 (amino acid residues from about 126 to about 147 in SEQ ID NO:2);
(j) the amino acid sequence of the calcitonin receptor intracellular domain 5 (amino acid residues from about 160 to about 187 in SEQ ID NO:2);
(k) the amino acid sequence of the calcitonin receptor transmembrane domain 6 (amino acid residues from about 209 to about 230 in SEQ ID NO:2);
(l) the amino acid sequence of the calcitonin receptor transmembrane domain 7 (amino acid residues from about 237 to about 258 in SEQ ID NO:2); and
(m) the amino acid sequence of an epitope-bearing portion of any one of the peptides of (a), (b), (c), (d), (e), (f), (g), (h), (i), (j), (k), or (l).

11. An isolated polypeptide comprising an epitope-bearing portion of any of the calcitonin receptor, wherein said portion is selected from the group consisting of: a polypeptide comprising amino acid residues from about 10 to about 20 in SEQ ID NO:2; a polypeptide comprising amino acid residues from about 49 to about 60 in SEQ ID NO:2; a polypeptide comprising amino acid residues from about 113 to about 123 in SEQ ID NO:2; a polypeptide comprising amino acid residues from about 145 to about 154 in SEQ ID NO:2; a polypeptide comprising amino acid residues from about 189 to about 209 in SEQ ID NO:2; and a polypeptide comprising amino acid residues from about 259 to about 560 in SEQ ID NO:2.

12. An isolated antibody that binds specifically to a calcitonin receptor polypeptide of claim 10.

13. An isolated nucleic acid molecule comprising a polynucleotide which hybridizes under stringent hybridization conditions to a polynucleotide having a nucleotide sequence identical to a nucleotide sequence in (a), (b), (c), (d) (e), (f), (g), (h), (i), (j), (k), (l) or (m) in claim 1 wherein said polynucleotide which hybridizes does not hybridize under stringent hybridization conditions to a polynucleotide having a nucleotide sequence consisting of only A residues or of only T residues.

14. An isolated nucleic acid molecule comprising a polynucleotide encoding an calcitonin receptor polypeptide wherein, except for at least one to fifty conservative amino acid substitutions, said polypeptide has a sequence selected from the group consisting of:
(a) the amino acid sequence of the calcitonin receptor polypeptide comprising the amino acid sequence from about 1 to about 568 in SEQ ID NO:2;
(b) the amino acid sequence of the calcitonin receptor polypeptide comprising the amino acid sequence from about 2 to about 568 in SEQ ID NO:2;
(c) the amino acid sequence of the calcitonin receptor polypeptide having the amino acid sequence encoded by the cDNA clone contained in ATCC Deposit No. 97730;
(d) the amino acid sequence of the calcitonin receptor extracellular regions selected from the group consisting of: amino acid residues from about 1 to about 18, amino acid residues from about 148 to about 159, amino acid residues from about 188 to about 209, and amino acid residues from about 231 to about 236 in SEQ ID NO:2;
(e) the amino acid sequence of the calcitonin receptor intracellular regions selected from the group consisting of:amino acid residues from about 50 to about 59, amino acid residues from about 110 to about 125, amino acid residues from about 188 to about 209, and amino acid residues from about 259 to about 560 in SEQ ID NO:2;
(f) the amino acid sequence of the calcitonin receptor transmembrane domain 1 (amino acid residues from about 20 to about 49 in SEQ ID NO:2);
(g) the amino acid sequence of the calcitonin receptor transmembrane domain 2 (amino acid residues from about 60 to about 80 in SEQ ID NO:2);
(h) the amino acid sequence of the calcitonin receptor transmembrane domain 3 (amino acid residues from about 82 to about 109 in SEQ ID NO:2);
(i) the amino acid sequence of the calcitonin receptor transmembrane domain 4 (amino acid residues from about 126 to about 147 in SEQ ID NO:2);
(j) the amino acid sequence of the calcitonin receptor intracellular domain 5 (amino acid residues from about 160 to about 187 in SEQ ID NO:2);
(k) the amino acid sequence of the calcitonin receptor transmembrane domain 6 (amino acid residues from about 209 to about 230 in SEQ ID NO:2);
(l) the amino acid sequence of the calcitonin receptor transmembrane domain 7 (amino acid residues from about 237 to about 258 in SEQ ID NO:2); and
(m) the amino acid sequence of an epitope-bearing portion of any one of the peptides of (a), (b), (c), (d), (e), (f), (g), (h), (i), (j), (k) or (l).

15. An isolated antibody that binds specifically to a calcitonin receptor polypeptide of claim 14.

16. A method of treating abnormal conditions related to an excess of calcitonin receptor activity comprising administering to a patient in need there of an antagonist of the polypeptide of claim 14.

17. A method of treating abnormal conditions related to an under expression of calcitonin receptor activity comprising administering to a patient in need there of an angonist of the polypeptide of claim 14.

18. A method of treating diseases or disorders selected from the group consisting of bacterial, fungal, protozoan and viral infections; infection caused by HIV-1 or HIV-2; pain; cancers; anorexia; bulimia; asthma; Parkinson's disease; acute heart failure; hypotension; hypertension; urinary retention; osteoporosis; angina pectoris; myocardial infarction; ulcers; allergies; benign prostatic hypertrophy; psychotic and neurological disorders, including anxiety, schizophrenia, manic depression, delirium, dementia or severe mental retardation; and dyskinesias comprising administering to a patient in need thereof an effective amount of an antagonist of the the polypeptide of claim 14.
